(19) United States
(12) Patent Application Publication
(10) Pub. No.: US 2007/0276437 A1
(43) Pub. Date: Nov. 29, 2007

(54) LOCKERS FOR SURGICAL TENSIONING MEMBERS AND METHODS OF USING THE SAME TO SECURE SURGICAL TENSIONING MEMBERS

(75) Inventors: Aaron M. Call, Lowell, MA (US); Karl R. Leinsing, Hampton, NH (US); Paul T. Modoono, Chelmsford, MA (US); Hiroatsu Sugimoto, Cambridge, MA (US); Edward I. McNamara, Chelmsford, MA (US); Joseph P. Lane, Methuen, MA (US); Matthew J. Murphy, Braintree, MA (US); Michael R. Sansoucy, Fiskdale, MA (US); Steven D. Cahalane, Pelham, NH (US); Christopher C. Lee, Tewksbury, MA (US); Jason H. Robinson, Salem, NH (US)

Correspondence Address: WOOD, HERRON & EVANS, LLP, 2700 CAREW TOWER, 441 VINE STREET, CINCINNATI, OH, 45202, US

(73) Assignee: MITRALIGN, INC., Tewksbury, MA (US)

(21) Appl. No.: 11/753,921

(22) Filed: May 25, 2007

Related U.S. Application Data
(60) Provisional application No. 60/803,183, filed on May 25, 2006.

(51) Int. Cl. A61B 17/04

(52) U.S. Cl. 606/232

(57) ABSTRACT

A locker for securing one or more tensioning members includes a locker body having a first aperture, a second aperture, and a passageway extending therebetween. A pin is coupled to the locker body so as to traverse the passageway and is movable between a latent condition wherein the tensioning members are movable with respect to the locker body, and an activated condition wherein the tensioning members are prevented from moving relative to the locker body. A method of using a suture locker includes delivering the suture locker to a surgical site via a catheter assembly and actuating the pin so as to move the locker from the latent condition to the activated condition.

### CROSS REFERENCE TO RELATED APPLICATIONS

This application claims priority to provisional patent application Ser. No. 60/803,183 filed on May 25, 2006, the disclosure of which is expressly incorporated by reference herein in its entirety.

### FIELD OF THE INVENTION

The present invention generally relates to lockers for use during catheter-based surgical procedures and, more particularly, to lockers and methods of using such lockers to secure one or more tensioning members, such as sutures, extending from corresponding attachment points with biological tissue, such as during the performance of an annuloplasty procedure.

### BACKGROUND OF THE INVENTION

Catheter-based surgical procedures may be employed to repair a defective mitral valve. One such catheter-based surgical procedure, commonly referred to as an annuloplasty, reduces the length of a posterior mitral valve leaflet through one or more plications. To that end, anchors are secured at a plurality of locations distributed about the annulus near the posterior leaflet of the mitral valve. Each anchor has a suture coupled thereto, which are collectively gathered and pulled tight. As the sutures are pulled tight, the tissue between each pair of adjacent anchors is plicated, thereby shortening the length of the annulus and drawing the posterior leaflet toward the anterior leaflet to effect mitral valve repair.

The suture for each anchor extends back within the catheter. To preserve the plications, the sutures must be secured against movement. Because the procedures are catheter based, suture lockers are typically used because the small diameter of the cannula may prohibit knotting.

There is generally a need for an improved locker to secure one or more tensioning members, such as sutures, against relative movement during a catheter-based surgical procedure.

### SUMMARY OF THE INVENTION

Embodiments of the invention comprise a locker for securing one or more tensioning members including a locker body and a means coupled to the locker body for securing the tensioning members. The means defines a latent condition wherein the tensioning members are movable relative to the locker body, and an activated condition wherein the tensioning members are prevented from moving relative to the locker body. In one embodiment, the locker body includes a first aperture, a second aperture, and a passageway extending therebetween. The tensioning members are threaded through the passageway of the locker body. A pin is coupled to the locker body and is movable in a direction generally between the first and second apertures. The pin defines a first position within the passageway in which the tensioning members are movable relative to the locker body and a second position within the passageway in which the tensioning members are substantially secured against movement relative to the locker body.

In another embodiment, the locker body includes a first aperture, a second aperture, and a passageway extending therebetween for receiving the tensioning members therethrough. A cam member is positioned within the passageway and is rotatable between a latent condition and an activated condition. In the latent condition the tensioning members are movable relative to the locker body and in the activated condition the tensioning members are prevented from moving relative to the locker body. The tensioning members may be secured against movement relative to the locker body through frictional engagement with the cam member. Additionally or alternatively, a biasing member may be coupled to the cam member to bias the cam toward the activated condition.

In another embodiment, the locker body includes a first aperture, a second aperture, and a passageway extending therebetween for receiving the tensioning members therethrough. The locker body includes a pair of opposed slots that receive a first pin and a second pin, at least one of the pins being capable of movement along the slots so as to define a latent condition wherein the tensioning members are movable relative to the locker body, and an activated condition wherein the pins pinch the tensioning members therebetween so as to prevent movement of the tensioning members relative to the locker body. One of the pins may be fixed relative to the slots or both pins may be movable along the slots.

In another embodiment, the locker body includes a first aperture, a second aperture, and a passageway extending therebetween for receiving the tensioning members therethrough. At least one spring arm is positioned in the passageway and is movable between a latent condition wherein the tensioning members are movable relative to the locker body and an activated condition wherein the tensioning members are prevented from moving relative to the locker body. The spring arm may be biased toward the activated condition and may be configured as a one-way locker wherein the spring arm resiliently yields to permit movement of the tensioning members in a first direction and opposes movement of the tensioning members in a second direction opposite the first direction. In one embodiment, two spring arms may be axially spaced along the locker body while in another embodiment, two spring arms may oppose each other so as to capture the tensioning members between the two spring arms. In still another embodiment, the spring arm may include a ring portion position in an aperture of the locker body wherein the ring portion cooperates with the walls of the aperture to capture the tensioning members therebetween.

In another embodiment, the locker body includes at least one passageway with the tensioning members disposed therein. In one aspect, two such passageways are provided wherein a pulling member pulls the tensioning members from one passageway and into another so as to create an interference fit so as to prevent the tensioning members from moving relative to the locker body. In another aspect, the locker body may be pulled inside a lumen of a tubular member to create a friction fit to prevent the tensioning members from moving relative to the locker body.

In another embodiment, the locker body includes a first aperture, a second aperture, and a passageway extending therebetween for receiving the tensioning members therethrough. A plurality of clamping arms are separated by the passageway and are movable relative to each other between a first position wherein the tensioning members are movable relative to the locker body and a second position wherein the tensioning members are clamped between the clamping arms to prevent movement of the tensioning members relative to the locker body. In one aspect, the locker body includes a shape memory alloy exhibiting a shape memory effect that provides a transition from the first position to the second position. In another aspect, the clamping arms may be biased toward the second position and a removable separating member used to maintain the clamping arms in the first position.

In still another embodiment, an outer locker body may include a first aperture, a second aperture, and a passageway extending therebetween for receiving the tensioning members therethrough. An inner expandable member may be disposed in the passageway which is movable between a latent condition wherein the tensioning members are movable relative to the locker body and an activated condition wherein the tensioning members are prevented from moving relative to the locker body. In one such embodiment, the expandable member may be a coil spring. In another such embodiment, the expandable member may be an expandable mesh tubular member such as a stent-like member.

### BRIEF DESCRIPTION OF THE DRAWINGS

FIG. 1 is an elevational view of a suture locker constructed in accordance with one embodiment of the invention in which the suture locker is shown in the activated position for locking tensioning members.

FIG. 1A is an exploded view of the suture locker shown in FIG. 1.

FIGS. 1B-1E are elevational views illustrating the operation of the suture locker shown in FIG. 1 and with the sutures omitted for clarity.

FIGS. 1F and 1G are cross-sectional views illustrating the operation of the suture locker shown in FIG. 1 and shown with the sutures threaded through the suture locker.

FIG. 1H is a cross-sectional view similar to FIGS. 1F and 1G of a suture locker constructed in accordance with an alternative embodiment of the invention.

FIGS. 1I and 1J are cross-sectional views illustrating the operation of a suture locker constructed in accordance with an alternative embodiment of the invention and shown with the sutures threaded through the suture locker.

FIGS. 1K-1M are elevational views illustrating the operation of the suture locker shown in FIG. 1 in accordance with an alternate embodiment.

FIG. 2A is an elevational view similar to FIG. 1 of a suture locker in accordance with another embodiment of the invention.

FIGS. 2B, 2C and 2D are a side, bottom and top views, respectively, of the suture locker shown in FIG. 2A.

FIG. 2E is a cross-sectional view of the suture locker of FIGS. 2A-2D shown with sutures threaded through the suture locker and the suture locker in a condition in which the sutures are movable through the suture locker.

FIG. 2F is a cross-sectional view similar to FIG. 2E but illustrating the sutures locked against movement relative to the suture locker.

FIG. 3A is an exploded view of a suture locker in accordance with another embodiment of the invention.

FIG. 3B is an elevational view of the suture locker of FIG. 3A shown assembled and coupled with a delivery tip of a catheter.

FIG. 3C is a partial cross-sectional view of the suture locker of FIGS. 3A and 3B shown with sutures threaded through the suture locker and the suture locker in a condition in which the sutures are movable through the suture locker.

FIG. 3D is a cross-sectional view similar to FIG. 3C but illustrating the sutures locked against movement relative to the suture locker.

FIG. 4A is an elevational view of a suture locker in accordance with another embodiment of the invention.

FIGS. 4B and 4C are bottom and top views of the suture locker of FIG. 4A.

FIG. 4D is a partial cross-sectional view of the suture locker of FIGS. 4A-4C shown with sutures threaded through the suture locker and the suture locker in a condition in which the sutures are movable through the suture locker.

FIG. 4E is a partial cross-sectional view similar to FIG. 4D but illustrating the sutures locked against movement relative to the suture locker.

FIG. 5A is an elevational view of a suture locker in accordance with another embodiment of the invention.

FIG. 5B is a cross-sectional view of the suture locker of FIG. 5A shown with sutures threaded through the suture locker and the suture locker in a condition in which the sutures are movable through the suture locker.

FIG. 5C is a cross-sectional view similar to FIG. 5B but illustrating the sutures locked against movement relative to the suture locker.

FIG. 6A is a front elevational view of a suture locker in accordance with another embodiment of the invention.

FIG. 6B is a partial cross-sectional view of the suture locker of FIG. 6A shown with sutures threaded through the suture locker and the suture locker in a condition in which the sutures are movable through the suture locker.

FIG. 7A is an elevational view of a suture locker in accordance with another embodiment of the invention.

FIG. 7B is a partial cross-sectional view of the suture locker of FIG. 7A shown with sutures threaded through the suture locker and the suture locker in a condition in which the sutures are movable through the suture locker.

FIG. 7C is a cross-sectional view similar to FIG. 7B but illustrating the sutures locked against movement relative to the suture locker.

FIG. 8A is an elevational view of a suture locker in accordance with another embodiment of the invention.

FIG. 8B is a cross-sectional view of the suture locker of FIG. 8A shown with sutures threaded through the suture locker and the suture locker in a condition in which the sutures are movable through the suture locker.

FIG. 8C is a cross-sectional view similar to FIG. 8B but illustrating the sutures locked against movement relative to the suture locker.

FIG. 9A is an elevational view of a suture locker in accordance with another embodiment of the invention.

FIG. 9B is a side view of the suture locker of FIG. 9A shown with sutures threaded through the suture locker and the suture locker in a condition in which the sutures are movable through the suture locker.

FIG. 9C is a side view similar to FIG. 9B but illustrating the sutures locked against movement relative to the suture locker.

FIGS. 10A-10C are cross-sectional views of a suture locker in accordance with another embodiment of the invention in which the operation of the suture locker is shown.

FIG. 11 is an elevational view of a suture locker in accordance with another embodiment of the invention shown with sutures threaded through the suture locker and the suture locker in a condition in which the sutures are movable through the suture locker.

FIG. 12 is an elevational view of a suture locker in accordance with another embodiment of the invention shown with sutures threaded through the suture locker and the suture locker in a condition in which the sutures are movable through the suture locker.

FIGS. 13A and 13B are elevational views of a suture locker in accordance with another embodiment of the invention in which the operation of the suture locker is depicted.

FIG. 14A is an elevational view of a suture locker in accordance with another embodiment of the invention.

FIG. 14B is a side view of the suture locker of FIG. 14A.

FIGS. 14C-14E are cross-sectional views of the suture locker of FIGS. 14A-14B in which the operation of the suture locker is depicted.

FIG. 15A is an elevational view of a suture locker in accordance with another embodiment of the invention.

FIG. 15B is a top view of the suture locker of FIG. 15A.

FIG. 15C is a cross-sectional view of the suture locker of FIGS. 15A, 15B.

FIGS. 15D and 15E are cross-sectional views in which the operation of the suture locker of FIGS. 15A-C is depicted.

FIG. 16A is an elevational view of a suture locker in accordance with another embodiment of the invention.

FIG. 16B is a cross-sectional view of the suture locker of FIG. 16A.

FIGS. 16C and 16D are cross-sectional views in which the operation of the suture locker of FIGS. 16A-16B is depicted.

FIG. 17A is an elevational view of a suture locker in accordance with another embodiment of the invention.

FIG. 17B is an end view of the suture locker shown in FIG. 17A.

FIG. 17C is a cross-sectional view of the suture locker shown in FIG. 17A.

FIGS. 17D-17F are cross-sectional views of the suture locker of FIGS. 17A-17C in which the operation of the suture locker is depicted.

FIG. 18 is a cross-sectional view of a suture locker in accordance with another embodiment of the invention which is similar to the embodiment shown in FIGS. 17A-17F.

FIG. 18A is an elevational view of a suture locker in accordance with another embodiment of the invention similar to the embodiment shown in FIGS. 17A-17F.

FIGS. 18B and 18C are cross-sectional views of the suture locker of FIG. 18A in which the operation of the suture locker is depicted.

FIG. 19A is an elevational view of a suture locker in accordance with another embodiment of the invention shown coupled to a tip of a delivery catheter.

FIG. 19B is a side elevational view of the suture locker shown in FIG. 19A.

FIG. 19C is a top elevational view of the suture locker shown in FIG. 19A.

FIG. 19D is a cross-sectional view of the suture locker shown in 19C taken along line 19D-19D.

FIGS. 19E-19G are cross-sectional views of the suture locker of FIGS. 19A-19D in which the operation of the suture locker is depicted.

FIG. 19H is an elevational view of the suture locker of FIGS. 19A-19D depicting operation of the suture locker.

### DETAILED DESCRIPTION

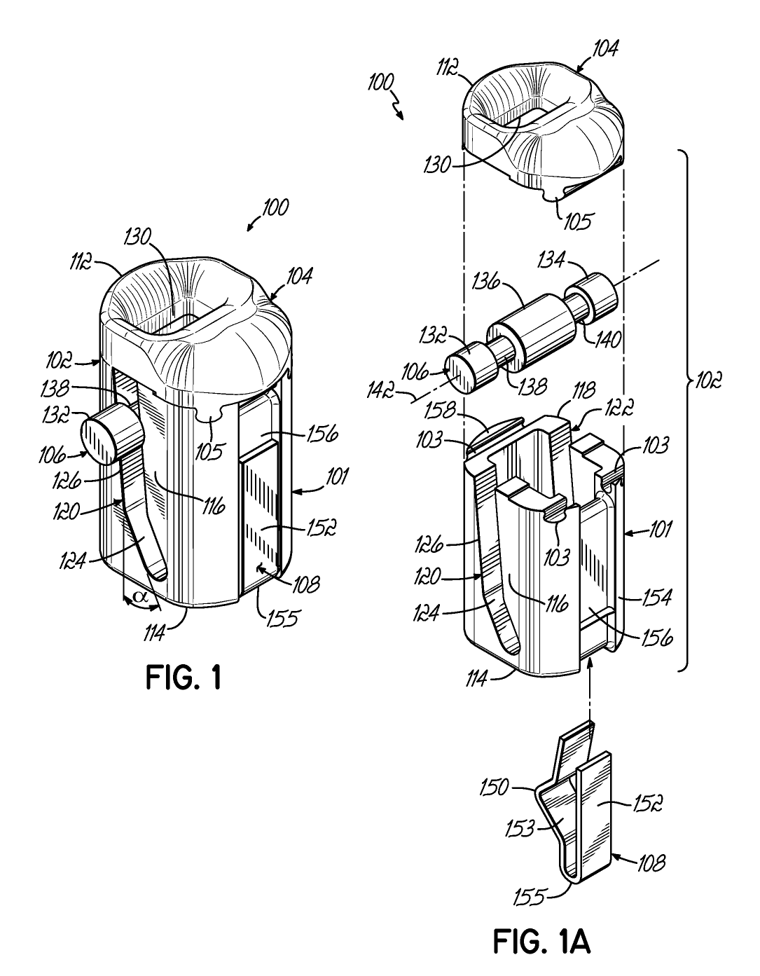

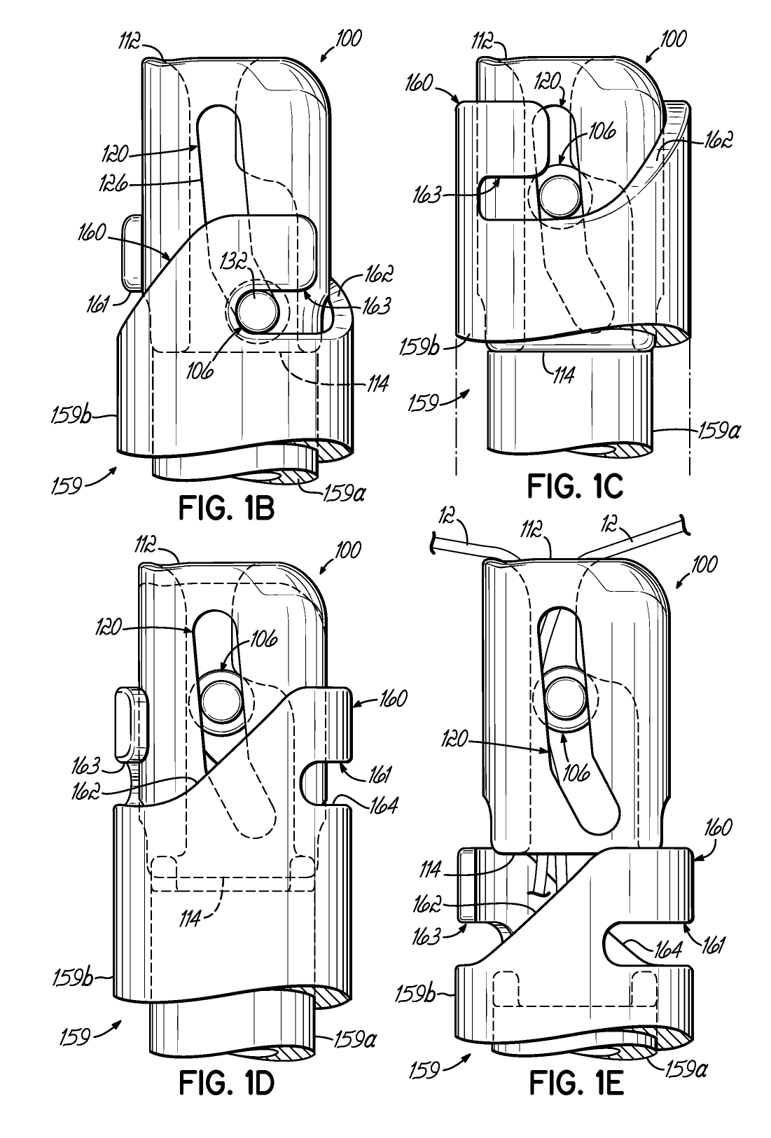

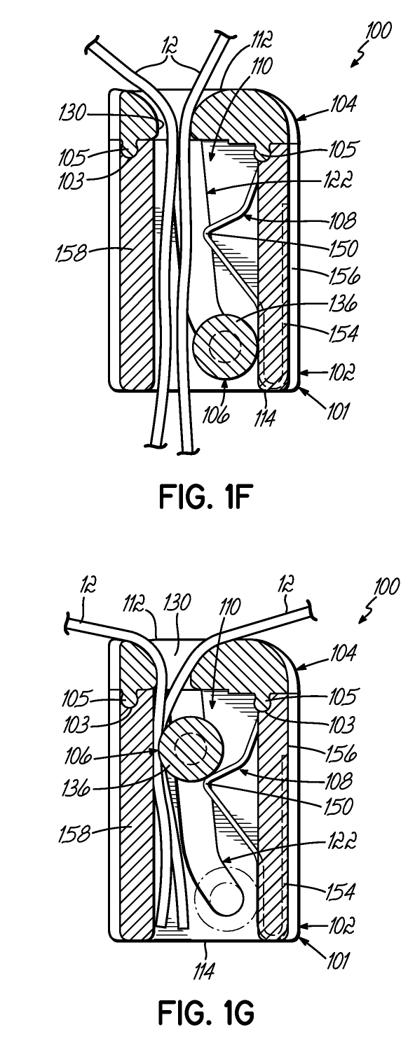

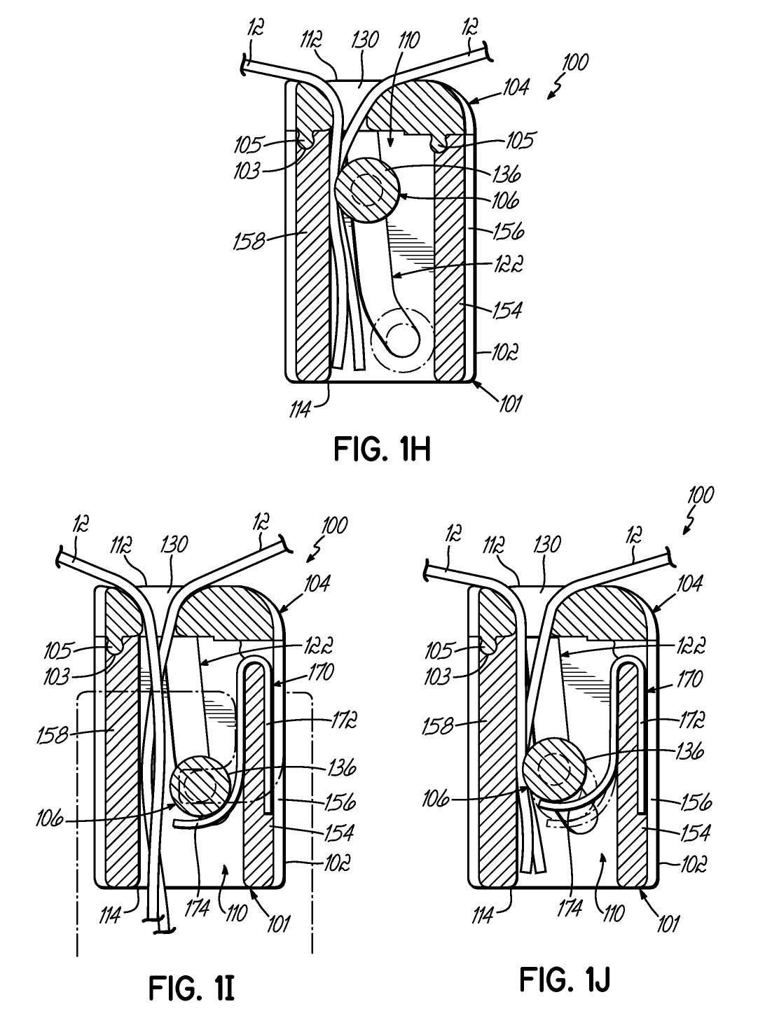

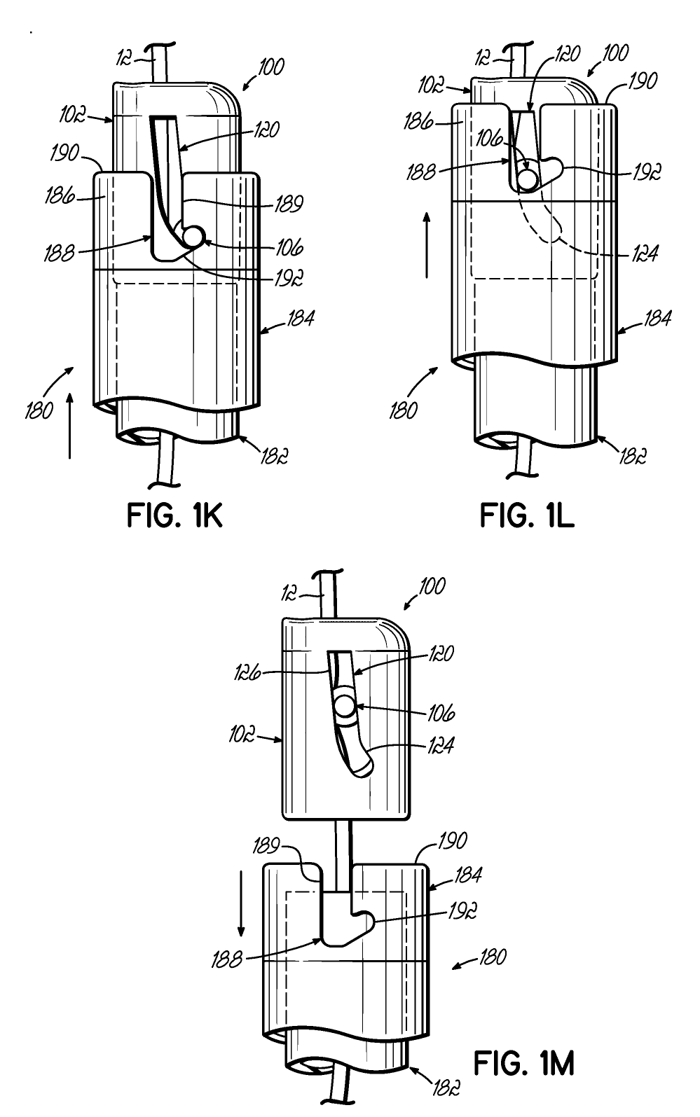

With reference to FIGS. 1 and 1A, a suture locker 100 constructed in accordance with a first embodiment of the invention is utilized to selectively captivate or capture one or more tensioning members 12 (FIGS. 1F and 1G) threaded through the suture locker 100. It will be appreciated that the tensioning members 12 may take other forms other than suture material, such as cable or any other small diameter flexible, semi-rigid or rigid member having a suitably high enough tensile strength for the intended use. Moreover, although we refer to embodiments of the invention as suture lockers, the invention contemplates that the suture lockers may be used with tensioning members other than sutures. The suture locker 100 has a first condition in which the tensioning members 12 are movable through the suture locker 100 and a second condition in which the tensioning members 12 are captivated and locked against movement with respect to the suture locker 100 and with respect to each other. The first condition may be referred to as a latent condition and the second condition may be referred to as an activated condition. Suture locker 100 may be used in conjunction with tensioning members extending from anchoring members shown and described in commonly-owned U.S. application Ser. No. 11/174,951. It is further appreciated that such a locker may have a plurality of passageways to introduce and capture sutures in this and other embodiments. Furthermore, although the suture lockers described herein may refer to a plurality of tensioning members extending therethrough, those of ordinary skill in the art will appreciate that, depending on the specific application, the suture lockers are also operative with a single suture.

Suture locker 100 comprises an assembly that includes a locker body 102 consisting of an assembly of a base member 101 and a contoured cap 104 that closes an open end of the base member 101, a pin 106, and a spring clip 108. The base member 101 and cap 104 constitute separate components of the assembly comprising the locker body 102 so that the pin 106 can be coupled with the locker body 102. To that end, a proximal edge of the cap 104 includes multiple projections 105 at spaced-apart locations that engage corresponding recesses 103 defined in a confronting distal edge of the locker body 102. When the suture locker 100 is assembled and in use, the cap 104 is located at a distal end of the suture locker 100 nearest to the biological tissue to which the tensioning members 12 are attached or anchored (not shown).

The locker body 102, which may be tubular and open ended, bounds a cavity or passageway 110 that extends from an aperture 114 at a proximal end of the locker body 102 toward the cap member 104. Opposite sidewalls 116, 118 of the base member 101 of locker body 102 include slots 120, 122, respectively, that are generally oriented in a proximal-distal direction and that communicate with the passageway 110. Slot 120 includes a relatively short segment 124 near the proximal aperture 114 of the locker body 102 and a relatively long segment 126 that intersects the short segment 124 at a shallow acute angle α (FIG. 1). The angle α is sufficient so that tensioning members 12 are movable through locker body 102 when the pin 106 is located in short segment 124. The orientation of the long segment 126 is nearer to being parallel with the proximal-distal direction. The long segment 126 extends from the intersection with the short segment 124 toward the distal end of the locker body 102. Similar considerations apply for slot 122 in sidewall 118, which is substantially identical to slot 120 in sidewall 116.

The cap 104 includes a passageway 130 that, when the suture locker 100 is assembled, permits access to the passageway 110 through a distal aperture 112. Passageway 130 smoothly merges with the passageway 110 in the locker body 102. The tensioning members 12 are threaded through the passageways 110, 130 of the suture locker 100. Contoured portions of the cap 102 extending from the distal aperture 112 toward the passageway 110 are smoothly curved inwardly in a convergent manner so that the cross-sectional area, when viewed along the proximal-distal direction, of passageway 130 narrows in a distal to proximal direction. The cross-sectional area of the passageway 130 is smaller than the corresponding cross-sectional area of passageway 110.

The cap 104, when assembled with the base member 101, closes the distal end of the slot 120. This constrains the movement of the pin 106 relative to the locker body 102 with a range of motion bounded by the opposite closed ends of the slot 120. Similar considerations apply for the bounding by cap 104 of slot 122 in sidewall 118, which is substantially identical to slot 120 in sidewall 116.

Pin 106 is constrained to move within the slot 120 along a path defined by the short and long segments 124, 126 and bounded in a proximal-distal direction by the opposite closed ends of the slot 120. The pin 106 includes peripheral sections or regions 132, 134, a central section or region 136, and intermediate or bridge regions 138, 140 that join a corresponding one of the peripheral regions 132, 134 to the central region 136. Each of the regions 132, 134, 136, 138, 140, which are arranged along a longitudinal axis 142 of the pin 106, is cylindrical in cross-sectional area when viewed along the longitudinal axis 142. Regions 138, 140 have the smallest diameter when viewed along the longitudinal axis 142 and region 136 has the largest diameter.

Bridge region 138 has a length along the longitudinal axis 142 that is determined primarily by the wall thickness of sidewall 116 of locker body 102. The diameter, or largest dimension, of bridge region 138 is selected to be smaller than the width of slot 120. The exposed faces of the peripheral region 132 and central region 136 bordering opposite sides of the sidewall 116, when the suture locker 100 is assembled and pin 106 is engaged with slots 120, 122, limit the transverse movement of the pin 106 in a direction parallel to the longitudinal axis 142 by contacting opposite confronting portions of the sidewall 116. Similar considerations apply for bridge region 140, which is substantially identical to bridge region 138 and is engaged with slot 122 in sidewall 118. The invention contemplates that the peripheral regions 132, 134 may be omitted from the construction of pin 106 while retaining the resistance against axial movement.

Spring clip 108, which is optional, is engaged with the locker body 102 in the assembly comprising the suture locker 100. The spring clip 108 includes an arm 153 and a shaped detent or spring element 150 that projects from arm 153 into the passageway 110 inside the tubular body 102 (FIGS. 1F and 1G). The spring clip 108 also includes an arm 152 that contacts an exterior surface of a sidewall 154, which connects sidewalls 116, 118, and a curved transition section 155 that joins arm 152 with arm 153 so that the arms 152, 153 are substantially parallel. The arms 152, 153 fit into a recess 156 that extends the length of the interior and exterior surfaces of sidewall 154 and wraps about the proximal edge of the bracket body 102. When the spring clip 108 is assembled with bracket body 102, the curved transition section 155 is disposed in the recess 156 proximate to the proximal edge.

Passageway 130 is offset transversely or laterally from an axis of symmetry extending through the passageway 110. As a result, passageway 130 is positioned closer to sidewall 158 of locker body 102 than sidewall 154, but is approximately centered in position between sidewalls 116, 118.

In use and with reference to FIGS. 1 and 1A-G, the bridge regions 138, 140 of pin 106 ride within the slots 120, 122. During the catheter-based surgical procedure, the tensioning members 12 may be threaded through the passageway 110 and passageway 130 of the suture locker 100 and are normally free to move with negligible resistance from the pin 106 between the distal and proximal apertures 112, 114 in the locker body 102 of suture locker 100, as depicted in FIGS. 1B and 1F. The pin 106 of the suture locker 100 is held initially in a latent condition relative to the locker body 102.

At an appropriate juncture of the catheter-based surgical procedure the distal end of the tensioning members 12 may be anchored to biological tissue at the procedure site and plications are formed by an axial force tensioning the tensioning members 12. At this point, the suture locker 100 may be activated to captivate the tensioning members 12. To that end, the suture locker 100 is coupled to a catheter assembly 159, which is maneuvered through the lumen of an outer sleeve (not shown) in the patient's vascular system to the surgical site as is generally known in the art. The catheter assembly 159 includes an inner catheter 159a to which the proximal end of the suture locker 100 is mated and an outer catheter 159b which is capable of moving relative to the inner catheter 159a (e.g., relative rotational movement). The tip of the outer catheter 159b includes an activation mechanism 160 for maintaining the suture locker 100 in the latent condition during deployment thereof and for activating the suture locker 100 so as to secure the tensioning members 12 thereto. The activation mechanism 160 may be manipulated or actuated to move the pin 106 to an activated condition to activate the suture locker 100 and capture the tensioning members 12. The activation mechanism 160 may then be manipulated to release its hold or grip on the suture locker 100 and be retracted from the surgical site.

The catheter assembly 159 delivers the suture locker 100 to the surgical site in a latent condition as shown in FIGS. 1B, 1F. Pin 106 is positioned such that the tensioning members 12 are moveable through the communicating passageways 110, 130. To place the suture locker in the activated condition, the outer catheter 159b and thus activation mechanism 160 is moved relative to the inner catheter 159a to displace the pin 106 within the slots 120, 122 in the distal direction toward the end cap 104. Contact between portions of the activation mechanism 160 bounding grooves or gaps 161, 163 and the peripheral regions 132, 134 of pin 106 supply an activation force for the suture locker. The pin 106 follows the contour of the slots 120, 122 as it moves from a latent condition shown in FIGS. 1B, 1F to an activated condition shown in FIGS. 1C, 1G.

As the pin 106 moves distally toward the distal end 112 of the locker body 102, the central region 136 traverses through the portion of the passageway 110 into which the shaped spring element 150 projects from arm 153 of spring clip 108. The central region 136 deflects or elastically deforms the spring element 150 in a direction toward wall 154 and then moves to a location distal to the spring element 150, as shown in FIG. 1G. After the deflection force applied by the central section 136 is removed, the deformed spring element 150 returns to its original undeflected condition, which blocks movement of the pin 106 in the proximal direction and captures the pin 106 in the headspace between the spring element 150 and the cap member 104. The pin 106 of the activated suture locker 100 pinches or compresses the tensioning members 12 between the central region 136 of pin 106 and a portion of the locker body 102 bordering the passageway 110.

As further shown in FIG. 1C and after the suture locker 100 is activated to captivate the tensioning members 12, the activation mechanism 160 of the outer catheter 159b is then rotated relative to the inner catheter 159a and the suture locker 100 coupled thereto so as to eliminate the engagement between the peripheral regions 132, 134 of pin 106 and the gaps 161, 163 defined in the activation mechanism 160. Continued rotation of the activation mechanism 160 relative to the inner catheter 159a decouples the suture locker 100 from the inner catheter 159a and from the activation mechanism 160, as depicted in FIG. 1D. The rotation of the activation mechanism 160 causes the peripheral regions 132, 134 to ride along a corresponding one of circumferentially-extending cam surfaces 162, 164, which may supply the force necessary to separate the suture locker 100 from the catheter assembly 159. The catheter assembly 159 may then be withdrawn in a proximal direction, as shown in FIG. 1E, to release the activated suture locker 100.

With reference to FIG. 1H and in accordance with an alternative embodiment of the invention, the spring clip 108 may be omitted from the suture locker 100. When pin 106 is moved from the latent condition (indicated in dot-dashed lines) to the activated condition, the interference between the tensioning members 12, the pin 106, and the interior surface of the locker body 102 cooperate to captivate the tensioning members 12. This eliminates the need for a blocking member in the construction of the suture locker 100.

With reference to FIGS. 1I and 1J and in accordance with an alternative embodiment of the invention, suture locker 100 may be provided with a biasing member 170, which replaces the spring clip 108 (FIGS. 1A-G). A portion 172 of the biasing member 170 is engaged with the locker body 102. Another portion 174 of the biasing member 170 defines an arm that contacts the pin 106. When the suture locker 100 is held in the latent condition by the activation mechanism 160, the pin 106 is held fixed against the biasing force applied by the biasing member 170.

Biasing member 170 may be formed from a superelastic material such as nitinol (NiTi), which has various advantages relating to the superelastic behavior. Specifically, NiTi superelastic materials have a relatively large recoverable deformation, a low permanent set or residual deformation, high plateau stresses and ultimate tensile strength, and high potential energy storage capability. NiTi superelastic materials, which are biocompatible, exhibit a corrosion resistance similar to stainless steels. Generally, biasing member 170 is cold-worked and heat treated to provide the superelastic condition, as understood by a person having ordinary skill in the art. Those of ordinary skill in the art will recognize that the biasing member 170 may also be formed of biocompatible elastomerics or other suitable materials. Moreover, biasing member 170 may be constructed from a shape memory alloy capable of exhibiting a shape memory effect.

FIGS. 1K-1M illustrate activation of the suture locker 100 in accordance with an alternate embodiment of the invention. As noted above in reference to FIGS. 1B-1E, a catheter assembly 159 may be used to deploy and activate the suture locker 100. In that embodiment, the activation mechanism 160 of the outer catheter 159b moved relative to the inner catheter 159a to displace the pin 106 within the slots 120, 122 in the distal direction by contacting gaps 161, 163 in activation mechanism 160. Once the suture locker is in the activated condition, however, the activation mechanism 160 is rotated relative to the inner catheter 159a to disengage the suture locker 100 from the catheter assembly 159.

As shown in FIGS. 1K-1M, suture locker 100 may be coupled to a catheter assembly 180, which is maneuvered through the lumen of an outer sleeve (not shown) in the patient's vasculature system to the surgical site, as is generally known in the art. The catheter assembly 180 includes an inner catheter 182 to which the proximal end of the suture locker 100 may be engaged, and an outer catheter 184 which is capable of axial movement relative to the inner catheter 182. The tip of the outer catheter 184 includes an activation mechanism 186 for maintaining the suture locker 100 in the latent condition during deployment thereof and for activating the suture locker so as to secure the tensioning members 12 thereto in the manner described above. The activation mechanism 186 may be manipulated or actuated to move the pin 106 to an activated condition so as to activate the locker 100 and capture the tensioning members. Advantageously, manipulating the activation mechanism 186 to activate the suture locker 100 also disengages the locker 100 from the catheter assembly 180.

To this end, and as shown in FIG. 1K, the activation mechanism 186 includes a J-shaped slot 188 having a first leg 189 extending proximally from a distal end 190 thereof and a shorter second leg 192 forming an acute angle with respect to the first leg 189 and extending back toward the distal end 190 but stopping short thereof. The first leg 189 extends generally in the proximal-distal direction. The slot 188 is open at its distal end so as to receive the pin 106 therein, but is closed at its proximal end. The catheter assembly 180 delivers the suture locker 100 to the surgical site in a latent condition as shown in FIG. 1K. The pin 106 is positioned in the second leg 192 at the proximal end of J-shaped slot 188 and the tensioning members 12 are movable relative to the suture locker 100. To place the suture locker 100 in the activated condition, the outer catheter 184, and thus activation mechanism 186, is moved axially relative to the locker body 102 and/or the inner catheter 182 to displace the pin 106 within the slots 120, 122 in the distal direction toward end cap 104. Contact between the edges of slot 188 and the peripheral regions 132, 134 of pin 106 supply an activation force such that pin 106 follows the contour of slots 120, 122 to move from the latent condition shown in FIG. 1K to an activated condition shown in FIG. 1L.

In this embodiment, as the pin 106 traverses short segment 124, which is angled relative to long segment 126, the pin 106 moves along the slot 188 so as to be located in first leg 189 as the pin 106 begins to move along long segment 126. In other words, the shape of slots 120, 122 causes the pin 106 to move into the first leg 189 of slot 188 by moving the outer catheter 184 distally relative to the locker body 102 and/or inner catheter 182. Once the pin 106 is positioned in the first leg 189 of slot 188, the catheter assembly 180 need only be moved in the proximal direction to disengage the suture locker therefrom, as shown in FIG. 1M. Thus, relative axial movement moves the pin 106 to the activated condition and disengages the suture locker 100 from catheter assembly 180 such that no rotational movement is needed to accomplish the separation.

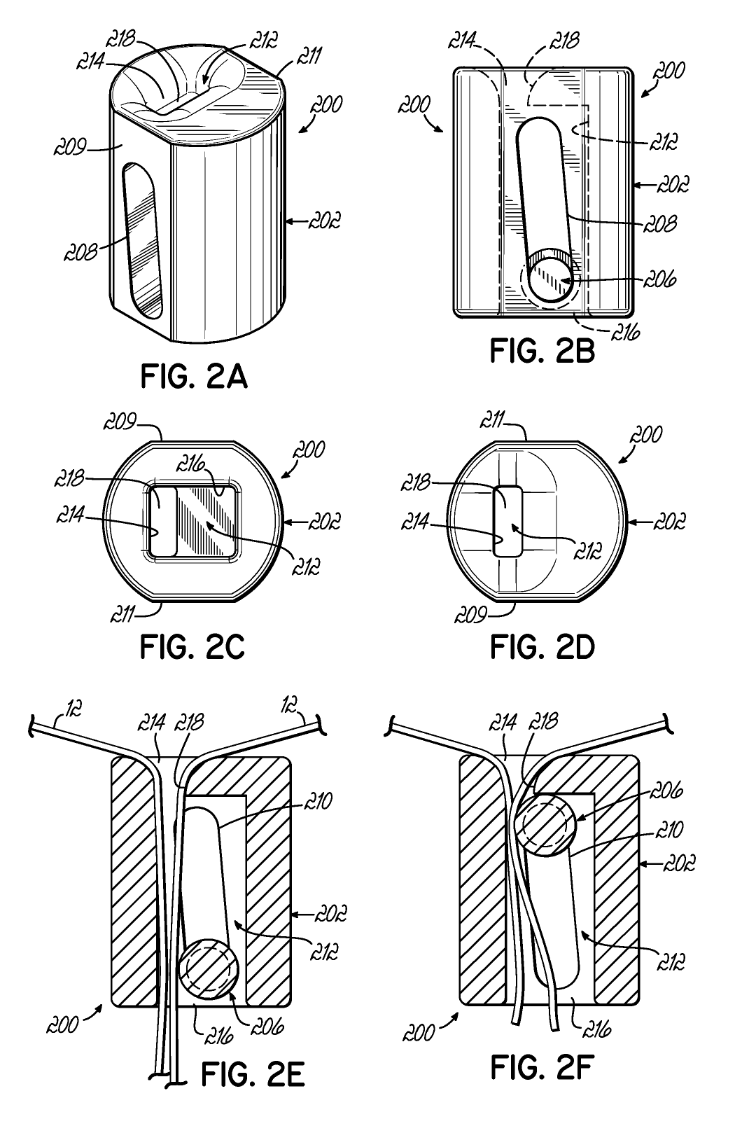

With reference to FIGS. 2A-2F and in accordance with an alternative embodiment of the invention, a suture locker 200 includes a locker body 202 and a pin 206 that rides in slots 208, 210 defined in the opposite sidewalls 209, 211 of the locker body 202. The construction and operation of the locker body 202, which has an integral cap, and the pin 206 are similar to the construction and operation of the locker body 102 and pin 106 of suture locker 100 (FIGS. 1, 1A-1G). Suture locker 200 also lacks a spring clip, similar to the embodiment of suture locker 100 depicted in FIG. 1H.

Locker body 202 includes a passageway 212 extending between distal and proximal apertures 214, 216 of the locker body 202. Passageway 212 includes a smaller passage 218 that communicates with the distal aperture 214. Slots 208, 210 are linear and not segmented with relatively inclined segments, as are slots 220, 222 (FIGS. 1, 1A-1G). Suture locker 200 has a latent condition, as best shown in FIG. 2E, and an activated condition, as best shown in FIG. 2F. The suture locker 200 may be deployed and activated using a catheter assembly similar to that described above for suture locker 100.

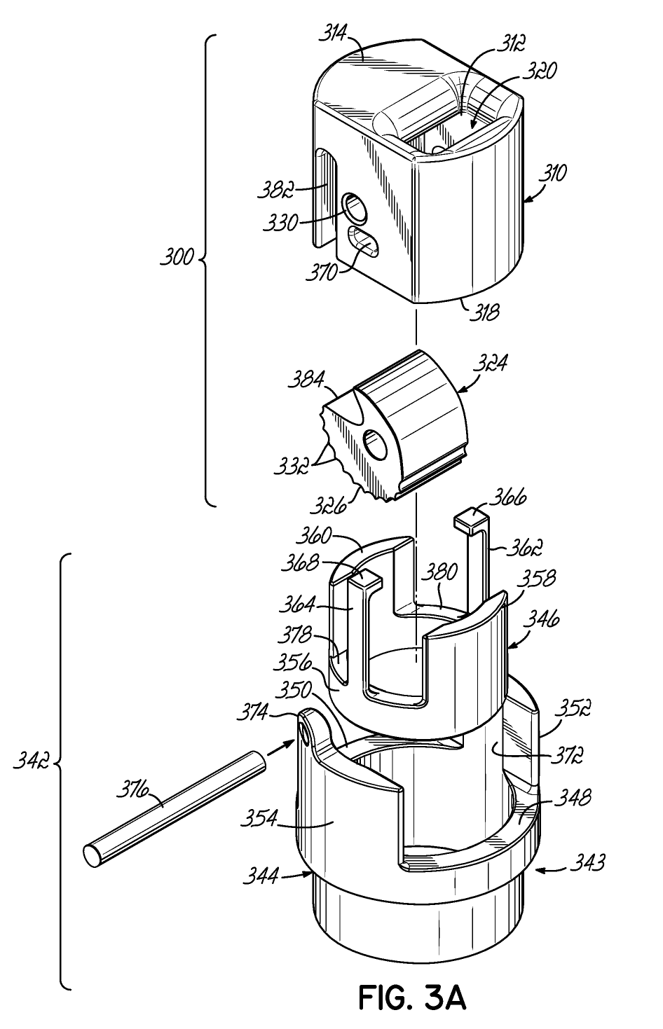

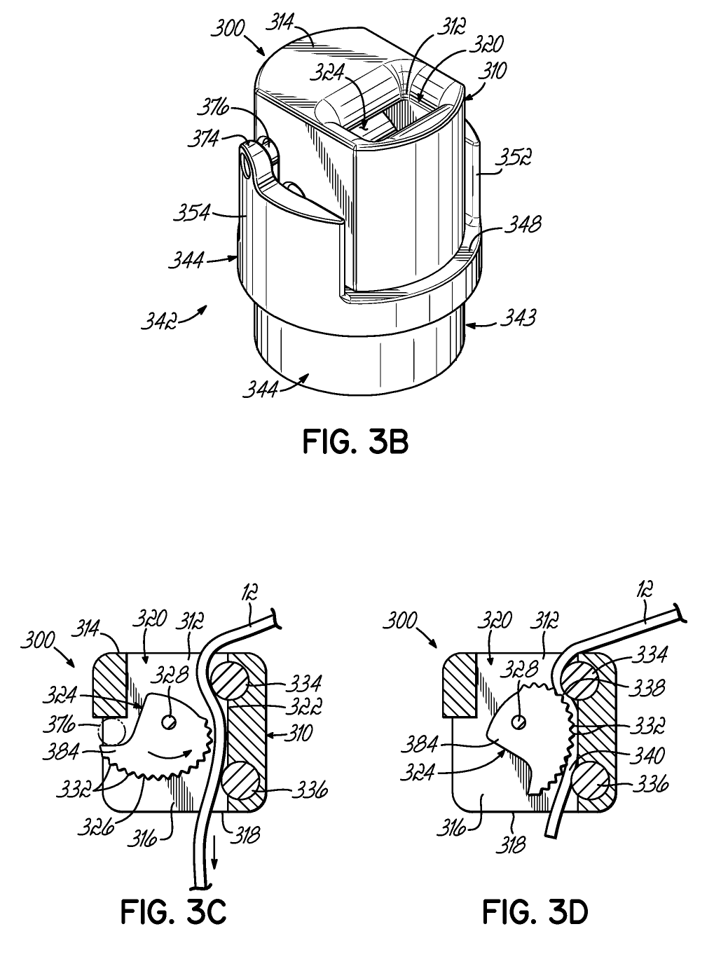

With reference to FIGS. 3A-3D and in accordance with an alternative embodiment of the invention, a suture locker 300 includes a locker body 310 having a contoured aperture 312 at a distal end 314 of locker body 310, a second aperture 316 at a proximal end 318 of locker body 310 and a passageway 320 extending between the proximal and distal apertures 312, 314 and through locker body 310. The passageway 320 defines an interior sidewall 322. A cam member 324 is rotatable relative to locker body 310 and includes a cam surface 326 adapted to engage one or more tensioning members 12 (FIGS. 3C and 3D). For instance, a pin 328 may be inserted through an aperture 330 in locker body 310 when cam member 324 is suitably positioned in passageway 320 and operates as a pivot point around which cam member 324 may rotate. The cam surface 326 may include a plurality of serrations or teeth 332 or other surface-roughing feature as known in the art to increase the friction between the cam surface 326 and the tensioning members 12.

As shown in FIGS. 3C and 3D, the cam member 324 is movable between an latent condition where the cam surface 326 is moved away from sidewall 322 and an activated condition where the cam surface 326 is closely adjacent sidewall 322. In the latent condition, as shown in FIG. 3C, the tensioning members 12 may freely move in the proximal-distal direction through passageway 320 without significant interference with cam member 326. In this way, the suture locker 300 may freely move relative to tensioning members 12 thereby permitting the suture locker 300 to be placed at a desired location relative to tensioning members 12 such as at the surgical site. In the activated condition, the cam member 324 may be rotated counterclockwise relative to its latent condition to effectively capture the tensioning members 12 between sidewall 322 and cam surface 326. When in the activated condition, the tensioning members 12 are “locked” in that the tensioning members 12 may not move relative to the suture locker 300 or move relative to each other. The sidewall 322 of passageway 320 may include protrusions 334 and 336 that define pinching or compressing points 338, 340 respectively for enhanced compression of the tensioning members 12 when the cam member 324 is in the activated condition.

The suture locker 300 as described above may operate as a one-way type of locker. As best illustrated in FIG. 3D, once in the activated condition, any further movement of the tensioning members 12 relative to locker body 310 in the distal direction causes the cam member 324 to further rotate in the counterclockwise direction which further increases the compressive force between the cam surface 326 and sidewall 322. Such movement then effectively further locks the tensioning members 12 from movement relative to locker body 310 and relative to each other. On the other hand, movement of the tensioning members 12 relative to locker body 310 in the proximal direction causes the cam member 324 to rotate in the clockwise direction, which decreases the compressive force between the cam surface 326 and sidewall 322, and with sufficient force, may move the cam member 324 to the latent condition. The tensioning members 12 may then move relative to the locker body 310 and each other, so as to, for example, adjust the suture locker 300.

Suture locker 300 as described above may be either an active locker or a passive locker. An active locker includes a biasing member (not shown) that biases the cam member 324 toward the activated condition. By way of example, cam member 324 may include a torsional spring that biases the cam member 324 toward the activated condition. In this way, any rotation of the cam member 324 in the clockwise direction must at least overcome the biasing force caused by the spring. Those of ordinary skill in the art will recognize other biasing members that operate to bias the cam member 324 toward the activated condition.

The invention is not limited to active lockers but may also be configured as passive lockers. Passive lockers are maintained in the actuated condition position by the friction between the tensioning members 12 and the contacting portions of the suture locker 300, i.e., the sidewall 322 and cam surface 326. In this way, any rotation of the cam member 324 in the clockwise direction must at least overcome the friction force between the tensioning members 12 and the suture locker 300. The passive lockers may be moved from the latent condition to the activated condition using several different methods. For instance, in one method, a pulling member (e.g., a pull wire) may be used to move the cam member 324 to the activated condition once the suture locker 300 is properly positioned. In another method, the tensioning members 12 may be tightly pulled so as to tension or over-tension the members 12. The tensioning members 12 may then be moved so as to engage the teeth 332 of cam member 24. For example, as shown in FIG. 3C, if the tensioning members 12 are moved left relative to the locker body 310, the tensioning members 12 may engage teeth 332. Once engaged, the tension in the tensioning members 12 may be released causing the cam member 324 to rotate in the counterclockwise direction to the activated condition. The cam member 324 would then be maintained in the activated condition by the friction between the tensioning members 12 and the suture locker 300 and any remaining tension in the tensioning members 12.

FIGS. 3A and 3B illustrate the distal tip of a catheter assembly 342 for delivery of the suture locker 300 in a catheter-based system. To this end, the catheter assembly 342 includes a catheter 343 having a tip including a first outer portion 344 that is telescopingly received over a second inner portion 346. The first outer portion 344 includes a pair of opposed notches 348, 350 that define opposed walls 352, 354. The second inner portion 346 includes a base 356 with a pair of upwardly extending opposed walls 358, 360 and a pair of upwardly extending spring arms 362, 364. The second portion 346 is received within first portion 344 such that the top of walls 358 and 360 is substantially level with or slightly below the floor of notches 348, 350. The spring arms 362, 364 extend slightly above walls 358, 360 and include inwardly directed detents 366, 368, respectively. Spring arms 362, 364 are adapted to engage the locker body 310 of suture locker 300.

To this end, locker body 310 includes a pair of apertures 370 (one shown) for engaging detents 366, 368 in spring arms 362, 364. As shown in FIG. 3B, this engagement selectively secures the suture locker 300 to the catheter assembly 342. When it is desired to release the suture locker 300 from the catheter assembly 342, the spring arms 362, 364 may be disengaged from the apertures 370 in locker body 310. In particular, the walls 352, 354 of first portion 344 include grooves 372 (one shown) having a tapered surface that are adapted to receive spring arms 362, 364 therein. The grooves 372 are tapered such that as the second portion 346 is received in first portion 344, the spring arms 362, 364 and their respective detents 366, 368 move inward so as to engage apertures 370 in locker body 310. When the suture locker 300 is to be deployed, the second portion 346 is moved distally relative to first portion 344 allowing spring arms 362, 364 to move outward along grooves 372. When the second portion is moved sufficiently in the distal direction, the detents 366, 368 disengage from the apertures 370 in locker body 310 and the suture locker 300 may be released from catheter assembly 342. The catheter assembly 342 may then be retracted from the surgical site.

The catheter assembly 342 may also be configured to move the cam member 324 from the latent condition to the activated condition. To this end, opposed walls 352, 354 of first outer portion 344 include projections 374 (one shown) that receive a pin 376, which extends between walls 352, 354. Pin 376 is configured to move along slots 378, 380 in second inner portion 346 defined between spring arms 362, 364, wall 360, and base 356 when first and second portions 344, 346 move relative to each other. As shown in FIG. 3B, pin 376 is also received in a slot 382 in locker body 310. Slot 382 extends generally in the proximal-distal direction and is open at the proximal end 318 of locker body 310. The opposed end of slot 382 is closed. As illustrated in FIGS. 3B and 3C, when the suture locker 300 is coupled to catheter assembly 342, the cam member 324 is in the latent condition with pin 376 positioned distally of an arm portion 384 of cam member 324. As the catheter assembly 342 is disengaged from suture locker 300 as explained above, the pin 376 moves in the proximal direction so as to engage arm portion 384 and rotate cam member 324 in the counterclockwise direction to the activated condition, as shown in FIG. 3D.

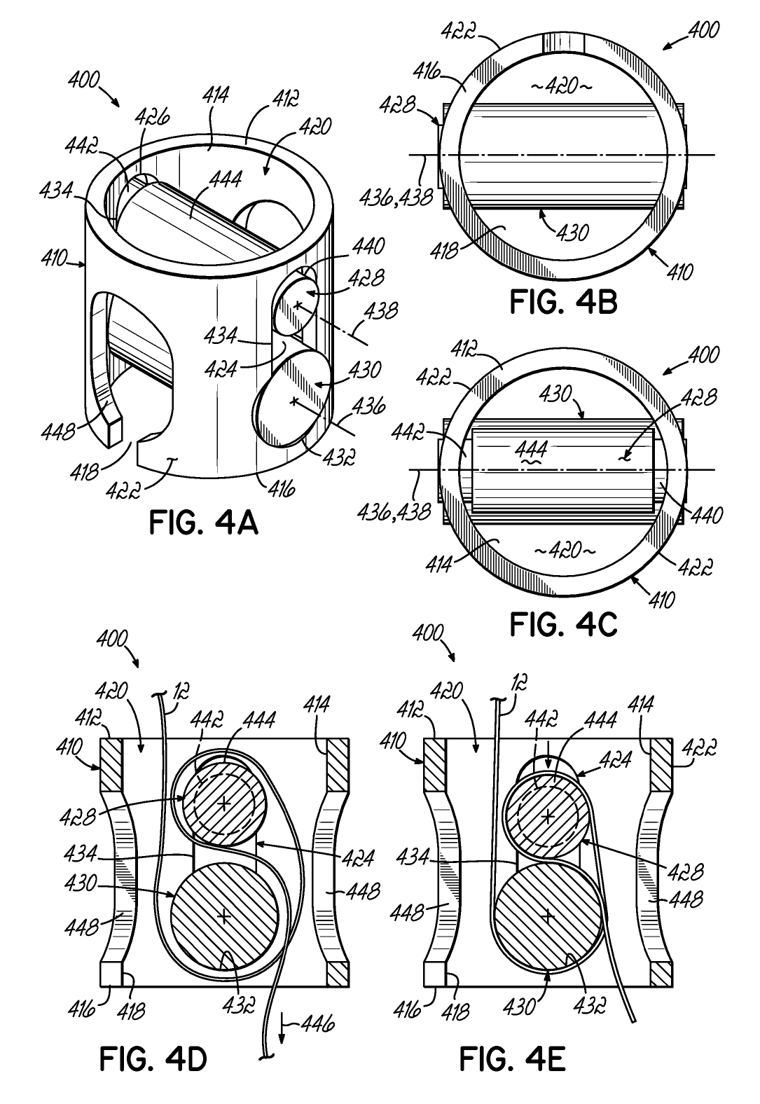

With reference to FIGS. 4A-4E and in accordance with an alternative embodiment of the invention, a suture locker 400 includes a generally cylindrical locker body 410 having a distal end 412 with an aperture 414 therein, a proximal end 416 with an aperture 418 therein, and a passageway 420 defined by sidewall 422 extending from the distal end 412 to proximal end 416 and through locker body 410. One or more tensioning members 12 may be disposed in the passageway 420, as depicted in FIGS. 4D and 4E. Sidewall 422 includes opposed slots 424, 426 extending generally in the proximal-distal direction and in which is disposed a pair of pins 428, 430. The slots 424, 426 terminate in closed ends short of proximal and distal ends 416, 412 of locker body 410. The slots 424, 426 have a keyhole shape with a proximal portion 432 enlarged relative to a narrower distal portion 434. Pin 430 is fixed within slots 424, 426 while pin 428 may be free to move within slots 424, 426. Pin 430 is symmetrical about a longitudinal axis 436 and is sized to be received in the enlarged proximal portion 432 of slots 424, 426. Pin 430 may be secured within the proximal portion 432 of slots 424, 426 through frictional engagement or other means such as welding, adhesive, etc. Pin 428 is symmetrical about a longitudinal axis 438 and includes peripheral regions 440, 442 and a central region 444 of larger relative cross-sectional area flanked along the longitudinal axis 438 by the peripheral regions 440, 442.

The diameter, or largest dimension, of the central regions 444 is larger than the width of the distal portion 434 of slots 424, 426 such that pin 428 is captured against lateral movement along the corresponding longitudinal axes 438 that would otherwise remove the pin 428 from the slots 424, 426. The diameter, or largest dimension, of the peripheral regions 440, 442 of pin 428 is smaller than the width of the distal portion 434 of slots 424, 426 such that the pin 428 is free to move within the slots 424, 426 between the closed end of the slots 424, 426 and pin 430.

The suture locker 400 has a latent condition, which is depicted in FIG. 4D, in which the tensioning members 12 are free to move in a proximal direction 446 substantially unhindered by the suture locker 400. The suture locker 400 further has an activated condition, which is depicted in FIG. 4E, in which the tensioning members 12 are captivated against movement in a proximal-distal direction 446 relative to the suture locker 400 and relative to each other. To this end, the tensioning members 12 may be pinched or compressed between the pins 428, 430.

In this regard, the suture locker 400 may be deployed and activated using a catheter assembly similar to catheter assembly 342 described above. In particular, the locker body 410 includes a pair of opposed apertures 448 adapted to receive a pair of spring arms on the tip of the catheter assembly to couple the suture locker 400 thereto. Additionally, the catheter assembly may include a pulling member (e.g., pull wire, suture, etc.) operatively coupled to pins 428, 430 so as to keep the suture locker 400 in the latent condition. When the tensioning members 12 have been suitably tensioned, the pulling member may be released so as to allow the pins 428, 430 to pinch the tensioning members 12 therebetween and place suture locker 400 in the activated condition. The suture locker 400 may then be released from the catheter assembly by releasing the spring arms from the apertures 448 similar to that described above. The catheter assembly may then be retracted from the surgical site.

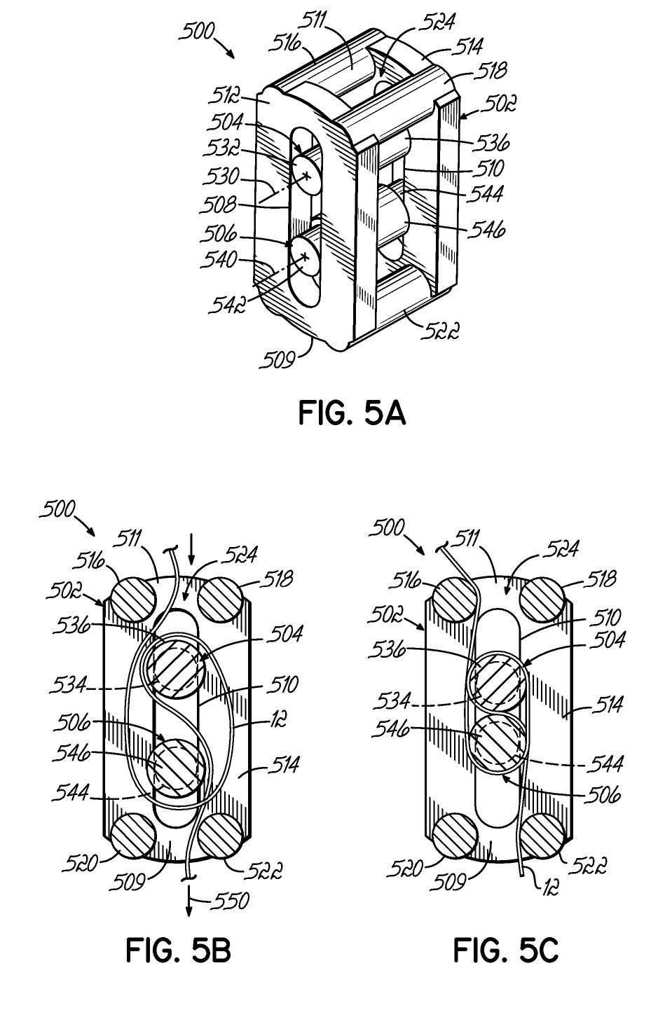

With reference to FIGS. 5A-5C and in accordance with an alternative embodiment of the invention, a suture locker 500 includes a locker body 502 and a pair of pins 504, 506 that ride in slots 508, 510 defined in sidewalls 512, 514, respectively, of the locker body 502. The slots 508, 510 terminate in closed ends disposed near proximal and distal apertures 509, 511 in the locker body 502. The sidewalls 512, 514 are coupled together at their respective corners by a plurality of cross members 516, 518, 520, 522, which are positioned and dimensioned such that a central passageway 524 extending through the locker body 502 between the proximal and distal apertures 509, 511 is not occluded. One or more tensioning members 12 may be disposed in the central passageway 524, as depicted in FIGS. 5B and 5C.

Pins 504, 506 are free to move within the slots 508, 510 relative to each other as constrained by the boundaries of the slots 508, 510, which are substantially aligned in a proximal-distal direction. Pin 504, which is symmetrical about a longitudinal axis 530, includes peripheral regions 532, 534 and a central region 536 of larger relative cross-sectional area flanked along the longitudinal axis 530 by the peripheral regions 532, 534. Similarly, pin 506, which is symmetrical about a longitudinal axis 540, includes peripheral regions 542, 544 and a central region 546 of larger relative cross-sectional area flanked along the longitudinal axis 540 by the peripheral regions 542, 544.

The diameter, or largest dimension, of the central regions 536, 546 is larger than the width of the slots 508, 510, respectively, such that the pins 504, 506 are captured against lateral movement along the corresponding longitudinal axes 530, 540 that would otherwise remove the pins 504, 506 from the slots 508, 510. The diameter, or largest dimension, of the peripheral regions 532, 534 of pin 504 and the peripheral regions 542, 544 of pin 506 are smaller than the width of the slots 508, 510 such that the pins 504, 506 are free to move within the slots 508, 510.

The tensioning members 12 are wound about the pins 504, 506, as shown in FIGS. 5B, 5C. The suture locker 500 has a latent condition, which is depicted in FIG. 5B, in which the tensioning members 12 are free to move about the pins 504, 506 in a proximal direction 550 substantially unhindered by the suture locker 500. In the latent condition, the separation between the pins 504, 506 within the passageway 524 permits substantially free movement of the tensioning members 12. The suture locker 500 has an activated condition, which is depicted in FIG. 5C, in which the tensioning members 12 are captivated against movement in a proximal-distal direction 550 relative to the suture locker 500. In the activated condition, the distance between the pins 504, 506 is reduced such that the tensioning members 12 are pinched or compressed between the pins 504, 506.

A catheter assembly (not shown) similar to catheter assembly 159 (FIGS. 1B-1E) may be used to deliver the suture locker 500 to the surgical site and to activate the suture locker 500 so as to capture the tensioning members 12 by reducing the distance between the pins 504, 506. Alternatively, the suture locker 500 may be deployed and activated using a catheter assembly having a catheter with a tip adapted to releasably couple with, for example, the proximal end of the locker body 502. The catheter assembly may further include a pulling member operatively coupled to pins 504, 506 so as to maintain the suture locker 500 in the latent condition. When the tensioning members 12 have been suitably tensioned, the pulling member may be released so as to allow the pins 504, 506 to pinch the tensioning members 12 therebetween to place the suture locker 500 in the activated condition. The suture locker 500 may then be released from the catheter assembly and the catheter assembly retracted from the surgical site.

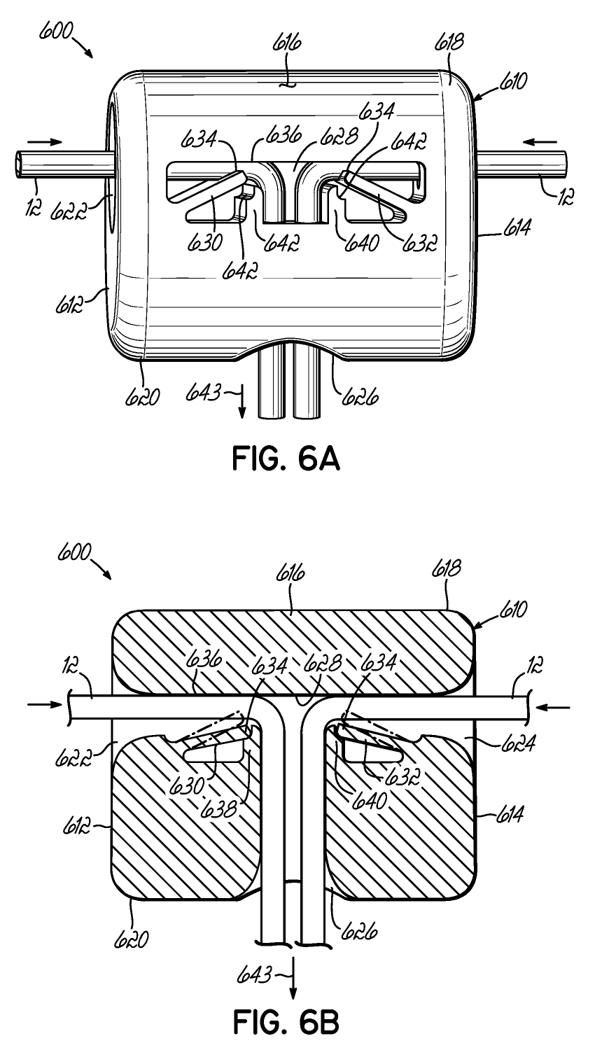

With reference to FIGS. 6A-6B and in accordance with an alternative embodiment of the invention, a suture locker 600 includes a generally cylindrical locker body 610 having first and second opposed end walls 612, 614, respectively, and a sidewall 616 that connects the end walls 612, 614 and defines a distal end 618 and a proximal end 620. The first end wall 612 includes a contoured aperture 622 therein adapted to receive one or more tensioning members 12. Likewise, second end wall 614 includes a contoured aperture 624 adapted to receive one or more tensioning members 12. The apertures 622, 624 in the end walls 612, 614 are located adjacent the distal end 618 of sidewall 616. The proximal end 620 of sidewall 616 also includes a contoured aperture 626. The suture locker 600 further includes a T-shaped passageway 628 through locker body 610 that connects apertures 622, 624 and 626. A first spring arm 630 may be positioned in the passageway 628 adjacent end wall 612 and a second spring arm 632 may be positioned in the passageway 628 adjacent end wall 614. Each of the spring arms 630, 632 includes a first end coupled to locker body 610 and a free end 634 projecting into the passageway 628 and toward each other. The free ends 634 may include a notch adapted to receive one or more tensioning members 12. The spring arms 630, 632 are angled in the distal direction so that the free ends 634 are adjacent an interior wall 636 defining passageway 628. The suture locker 600 may further include a pair of upstanding studs 638, 640 each having a recess 642 adapted to receive the free end 634 of spring arms 630, 632 when the spring arms 630, 632 are deflected in the proximal direction.

With reference to FIG. 6B, the distal end of each tensioning member 12 may be attached to tissue, such as with an anchor. Movement of the tensioning members 12 in a proximal direction 643 applies a force to the tensioning members 12 that may prompt the formation of one or more plications. To maintain the plications in the tissue, the movement of the tensioning members 12 relative to each other is restrained. To this end, the tensioning members 12 are threaded through apertures 622, 624 in opposed end walls 612, 614, through passageway 628, and out aperture 626 in the proximal end 620 of sidewall 616. As the tensioning members 12 are pulled relative to the suture locker 600 in the proximal direction, the spring arms 630, 632 deflect so as to move the free end 634 in the proximal direction away from interior wall 636 to a latent condition where movement of the tensioning members 12 through the suture locker 600 is permitted, as shown in FIG. 6B. The movement of the tensioning members 12 relative to the suture locker 600 may be what causes the deflection of the spring arms 630, 632. When there is no movement of the tensioning members 12 relative to the suture locker 600, the spring arms 630, 632 are biased in the distal direction so as to capture the tensioning members 12 between the free end 634 of spring arms 630, 632 and the interior wall 636 of the passageway 628. The suture locker 600 is in an activated condition and movement of the tensioning members 12 relative to the suture locker 600 and relative to each other is prevented.

The suture locker 600 as described above may be configured as a one-way type of locker. As best shown in FIG. 6A, once the suture locker 600 is in the activated condition, any movement of the tensioning members 12 relative to the suture locker 600 or relative to each other in the distal direction, which would result in loosening of the plications in the tissue, causes the spring arms 630, 632 to further deflect in the distal direction thereby increasing the compressive force that secures the tensioning members 12 with the suture locker 600. One engineering method of note involves beam deflection. Such movement then effectively further locks the tensioning members 12 from movement relative to the suture locker 600 or relative to each other in the distal direction. As explained above, however, the tensioning members 12 may be pulled in the proximal direction relative to the suture locker 600, which would in essence unlock the suture locker 600 and again permit movement of the tensioning members 12 relative to the suture locker 600 in the proximal direction.

The suture locker 600 may be deployed and activated using a catheter assembly having a catheter with a tip adapted to releasably couple with, for example, the proximal end 620 of the locker body 610. The catheter assembly may further include a pulling member operatively coupled to spring arms 630, 632 so as to maintain the suture locker 600 in the latent condition during deployment. When the tensioning members 12 have been suitably tensioned, the pulling member may be released so as to allow the spring arms 630, 632 to pinch the tensioning members 12 to place the suture locker in the activated condition. The suture locker 600 may then be released from the catheter assembly and the catheter assembly retracted from the surgical site.

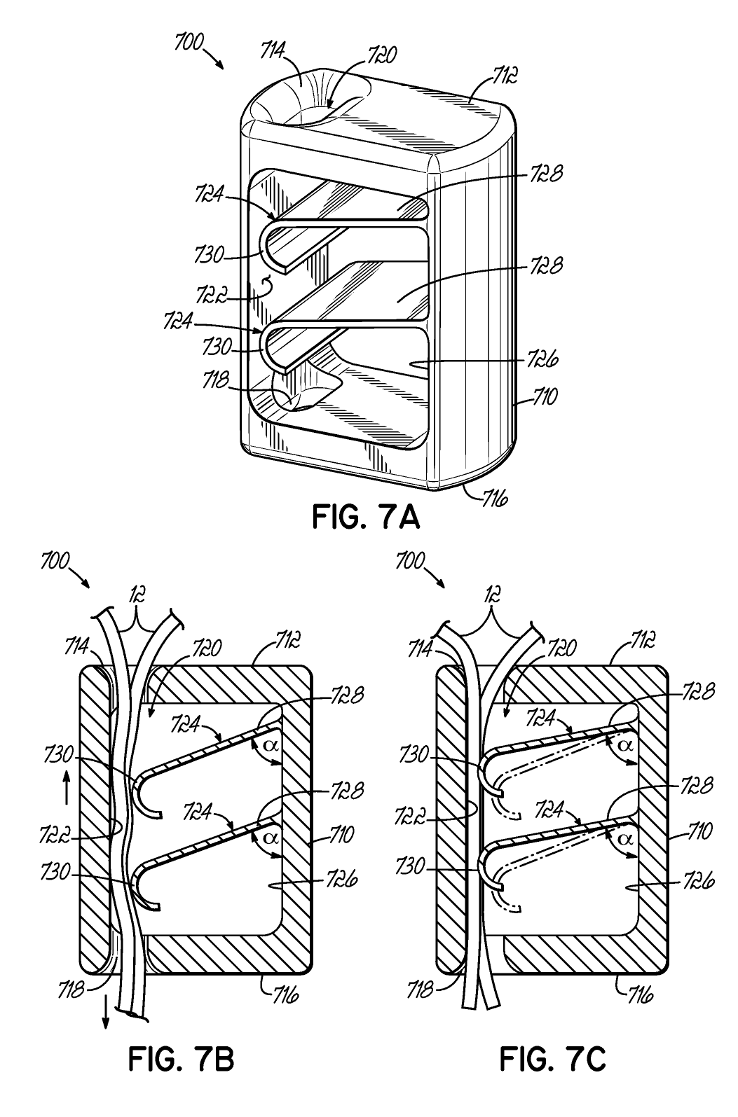

With reference to FIGS. 7A-7C and in accordance with an alternative embodiment of the invention, a suture locker 700 includes a generally rectangular locker body 710 having a distal end 712 having a contoured aperture 714 therein, a proximal end 716 having a contoured aperture 718 therein, and a passageway 720 extending between the apertures 714, 718 in distal and proximal ends 712, 716 and through locker body 710. The apertures 714, 718 are aligned in a proximal-distal direction and are positioned adjacent a first sidewall 722 of passageway 720. At least one (two shown in the figures) spring arms 724 project from a sidewall 726 of passageway 720 opposite sidewall 722 and toward sidewall 722. Each of the spring arms 724 includes a leg portion 728 and a U-shaped tip portion 730 that is closely adjacent sidewall 722. The spring arms 724 are angled toward the proximal end 716 of locker body 710 so as to define an acute angle a between the sidewall 726 and the leg portion 728 of the spring arms 724.

With reference to FIGS. 7B and 7C, the distal end of one or more tensioning members 12 may be attached to tissue, such as with an anchor. Movement of the tensioning members 12 in a proximal direction applies a force to the tensioning members 12 that may prompt the formation of one or more plications. To maintain the plications in the tissue, the movement of the tensioning members 12 relative to each other is restrained. To this end, the tensioning members 12 are threaded through apertures 714, 718 and through passageway 720. As the tensioning members 12 are pulled relative to the suture locker 700 in the proximal direction (e.g., either the suture locker 700 is held stationary and the tensioning members 12 are pulled in the proximal direction, or the tensioning members 12 are held stationary and the suture locker 700 is pushed in the distal direction), the spring arms 724 deflect in the proximal direction away from sidewall 722 defining a latent condition where movement of the tensioning members 12 through the suture locker 700 is permitted, as shown in FIG. 7B. The movement of the tensioning members 12 relative to the suture locker 700 may be what causes the deflection of the spring arms 724. When there is no movement of the tensioning members 12 relative to the suture locker 700, the spring arms 724 are biased toward the distal direction so as to capture the tensioning members 12 between the U-shaped tip portion 730 and the sidewall 722. The suture locker 700 is in an activated condition and movement of the tensioning members 12 relative to the suture locker 700 and relative to each other is prevented.

The suture locker 700 as described above may be configured as a one-way type of locker. As best shown in FIGS. 7A and 7B, once in the activated condition, any movement of the tensioning members 12 relative to the suture locker 700 or relative to each other in the distal direction (e.g., either the suture locker 700 is held stationary and the tensioning members 12 are pulled in the distal direction, or the tensioning members 12 are held stationary and the suture locker 700 is pulled in the proximal direction), which would result in loosening of the plication in the tissue, causes the spring arms 724 to further deflect in the distal direction and increase the compressive force that secures the tensioning members 12 with the suture locker 700. Such movement then effectively further locks the tensioning members 12 from movement relative to the suture locker 700 or relative to each other in the distal direction. As explained above, however, the tensioning members 12 may be pulled in the proximal direction relative to the suture locker 700, which would unlock the suture locker 700 and again permit movement of the tensioning members 12 relative to the suture locker 700 in the proximal direction.

The suture locker 700 may be deployed and activated using a catheter assembly having a catheter with a tip adapted to releasably couple with, for example, the proximal end 716 of the locker body 710. The catheter assembly may further include a pulling member operatively coupled to spring arms 724 so as to maintain the suture locker 700 in the latent condition during deployment. When the tensioning members 12 have been suitably tensioned, the pulling member may be released so as to allow the spring arms 724 to pinch the tensioning members 12 and place the suture locker in the activated condition. The suture locker 700 may then be released from the catheter assembly and the catheter assembly retracted from the surgical site.

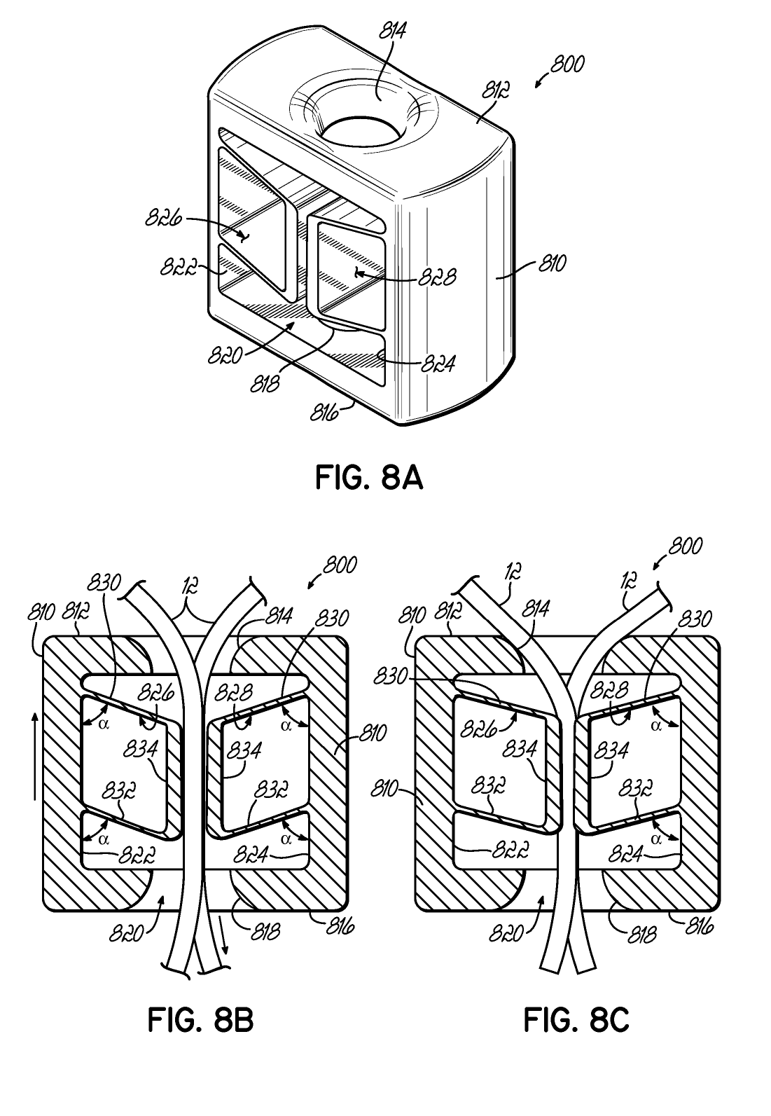

With reference to FIGS. 8A-8C and in accordance with an alternative embodiment of the invention, a suture locker 800 includes a generally rectangular locker body 810 having a distal end 812 with contoured aperture 814 therein, a proximal end 816 having a contoured aperture 818 therein, and a passageway 820 extending between the apertures 814, 818 in distal and proximal ends 812, 816 and through locker body 810. The apertures 814, 816 are aligned in a proximal-distal direction and are positioned approximately midway between a pair of opposed sidewalls 822, 824 that define passageway 820. A first spring arm 826 projects from sidewall 822 and toward sidewall 824 and a second spring arm 828 projects from sidewall 824 and toward sidewall 822. The spring arms 826, 828 have a similar construction thus only spring arm 826 will be described in detail. Spring arm 826 includes a pair of axially spaced leg portions 830, 832 and a connecting portion 834 that connects the ends of leg portions 830, 832. The connecting portion 834 is position adjacent the midline between the sidewalls 822, 824. The spring arms 826, 828 are angled toward the proximal end 816 of locker body 810 so as to define an acute angle a between the sidewalls 822, 824 and respective the leg portions 830, 832 of the spring arms 826, 828.

With reference to FIGS. 8B and 8C, the distal end of one or more tensioning members 12 may be attached to tissue, such as with an anchor. Movement of the tensioning members 12 in a proximal direction applies a force to the tensioning members 12 that may prompt the formation of one or more plications. To maintain the plications in the tissue, the movement of the tensioning members 12 relative to each other is restrained. To this end, the tensioning members 12 are threaded through apertures 814, 816 and through passageway 820. As the tensioning members 12 are pulled relative to the suture locker 800 in the proximal direction (e.g., either the suture locker 800 is held stationary and the tensioning members 12 are pulled in the proximal direction, or the tensioning members 12 are held stationary and the suture locker 800 is pushed in the distal direction), the spring arms 826, 828 deflect in the proximal direction away from each other defining a latent condition where movement of the tensioning members 12 through the suture locker 800 is permitted, as shown in FIG. 8B. The movement of the tensioning members 12 relative to the suture locker 800 may be what causes the deflection of the spring arms 826, 828. When there is no movement of the tensioning members 12 relative to the suture locker 800, the spring arms 826, 828 are biased toward the distal end 812 so as to capture the tensioning members 12 between the connecting portions 834 of the two spring arms 826, 828, as shown in FIG. 8C. The suture locker 800 is in an activated condition and movement of the tensioning members 12 relative to the suture locker 800 and relative to each other is prevented.

The suture locker 800 as described above may be configured as a one-way type of locker. As best shown in FIGS. 8B and 8C, once in the activated condition, any further movement of the tensioning members 12 relative to the suture locker 800 or relative to each other in the distal direction (e.g., either the suture locker 800 is held stationary and the tensioning members 12 are pulled in the distal direction, or the tensioning members 12 are held stationary and the suture locker 800 is pulled in the proximal direction), which would result in loosening of the plications in the tissue, causes the spring arms 826, 828 to further deflect in the distal direction thereby increasing the compressive force that secures the tensioning members 12 with the suture locker 800. Such movement then effectively further locks the tensioning members 12 from movement relative to the suture locker 800 or relative to each other in the distal direction. As explained above, however, the tensioning members 12 may be pulled in the proximal direction relative to the suture locker 800, which would in essence unlock the suture locker 800 and again permit movement of the tensioning members 12 relative to the suture locker 800 in the proximal direction.

The suture locker 800 may be deployed and activated using a catheter assembly having a catheter with a tip adapted to releasably couple with, for example, the proximal end 816 of the locker body 810. The catheter assembly may further include a pulling member operatively coupled to spring arms 826, 828 so as to maintain the suture locker 800 in the latent condition. When the tensioning members 12 have been suitably tensioned, the pulling member may be released so as to allow the spring arms 826, 828 to pinch the tensioning members 12 to place the suture locker in the activated condition. The suture locker 800 may then be released from the catheter assembly and the catheter assembly retracted from the surgical site.

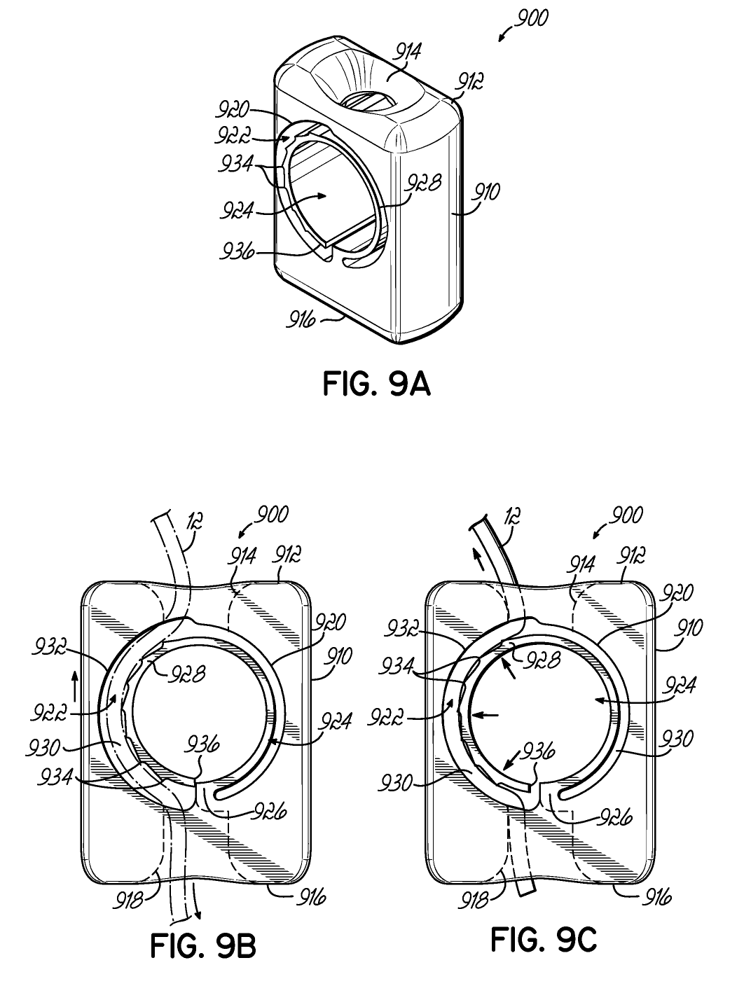

With reference to FIGS. 9A-9C and in accordance with an alternative embodiment of the invention, a suture locker 900 includes a generally rectangular locker body 910 having a distal end 912 having a contoured aperture 914 therein, and a proximal end 916 having a contoured aperture 918 therein. Suture locker 900 further includes a generally circular aperture 920 extending through locker body 910 so as to define a passageway 922 extending between apertures 914, 918 in distal and proximal ends 912, 916 and through locker body 910. The apertures 914, 916 are aligned along a central axis through locker body 910. A spring arm 924 is positioned within the aperture 920. The spring arm 924 includes a leg portion 926 and a generally circular ring portion 928. The leg portion 926 couples the spring arm 924 to the locker body 910. As shown in the figures, one end of the leg portion 926 is coupled to the locker body 910 adjacent the proximal end 916. The other end of leg portion 926 couples to the ring portion 928. The ring portion 928 is generally circular and is positioned within aperture 920. The diameter of ring portion 928 is less than the diameter of aperture 920 so as to define a small gap 930 between the outer surface of the ring portion 928 and an inner wall 932 defining passageway 922. The outer surface of the ring portion 928 includes a plurality of serrations or teeth 934 along at least a portion thereof. The teeth 934 are angled or slanted toward the proximal end 916 of the suture locker 900. The ring portion 928 includes a free end 936 adjacent leg portion 926 which allows at least a portion of the ring portion 928 to slightly expand or contract depending on the position of the free end 936.

With reference to FIGS. 9B and 9C, the distal end of one or more tensioning members 12 may be attached to tissue, such as with an anchor. Movement of the tensioning members 12 in a proximal direction applies a force to the tensioning members 12 that may prompt the formation of one or more plications. To maintain the plications in the tissue, the movement of the tensioning members 12 relative to each other is restrained. To this end, the tensioning members 12 are threaded through apertures 914, 916 and through passageway 922 so as to be in the gap 930 between the ring portion 928 of spring arm 924 and inner wall 932. As the tensioning members 12 are pulled relative to the suture locker 900 in the proximal direction (e.g., either the suture locker 900 is held stationary and the tensioning members 12 are pulled in the proximal direction, or the tensioning members 12 are held stationary and the suture locker 900 is pushed in the distal direction), the spring arm 924 deflects moving the free end 936 toward the leg portion 926 which effective contracts the ring portion 928 and defines a latent condition where movement of the tensioning members 12 through the suture locker 900 is permitted, as shown in FIG. 9B. The movement of the tensioning members 12 relative to the suture locker 900 may be what causes the deflection of the spring arm 924. When there is no movement of the tensioning members 12 relative to the suture locker 900, the spring arm 924 is biased outward expanding ring portion 928 so as to capture the tensioning members 12 between the outer surface of the ring portion 928 and the inner wall 932 of the passageway, as shown in FIG. 9C. The suture locker 900 is in an activated condition and movement of the tensioning members 12 relative to the suture locker 900 and relative to each other is prevented.

The suture locker 900 as described above may be configured as a one-way type of locker. As best shown in FIGS. 9B and 9C, once the suture locker 900 is in the locked activated condition, any movement of the tensioning members 12 relative to the suture locker 900 or relative to each other in the distal direction (e.g., either the suture locker 900 is held stationary and the tensioning members 12 are pulled in the distal direction, or the tensioning members 12 are held stationary and the suture locker 900 is pulled in the proximal direction), which would result in loosening of the plications in the tissue, causes the ring portion 928 of spring arm 924 to further expand as a result of the slanted configuration of the teeth 934 and the engagement of the teeth 934 with the tensioning members 12. The further expansion of ring portion 928 increases the compressive force that secures the tensioning members 12 with the suture locker 900. Such movement then effectively further locks the tensioning members 12 from movement relative to the suture locker 900 or relative to each other in the distal direction. As explained above, however, the tensioning members 12 may be pulled in the proximal direction relative to the suture locker 900, which would in essence unlock the suture locker 900 and again permit movement of the tensioning members 12 relative to the suture locker 900 in the proximal direction.

The suture locker 900 may be deployed and activated using a catheter assembly having a catheter with a tip adapted to releasably couple with, for example, the proximal end 916 of the locker body 910. The catheter assembly may further include a pulling member operatively coupled to spring arm 924 so as to maintain the suture locker 900 in the latent condition. When the tensioning members 12 have been suitably tensioned, the pulling member may be released so as to allow the spring arm 924 to pinch the tensioning members 12 to place the suture locker in the activated condition. The suture locker 900 may then be released from the catheter assembly and the catheter assembly retracted from the surgical site.

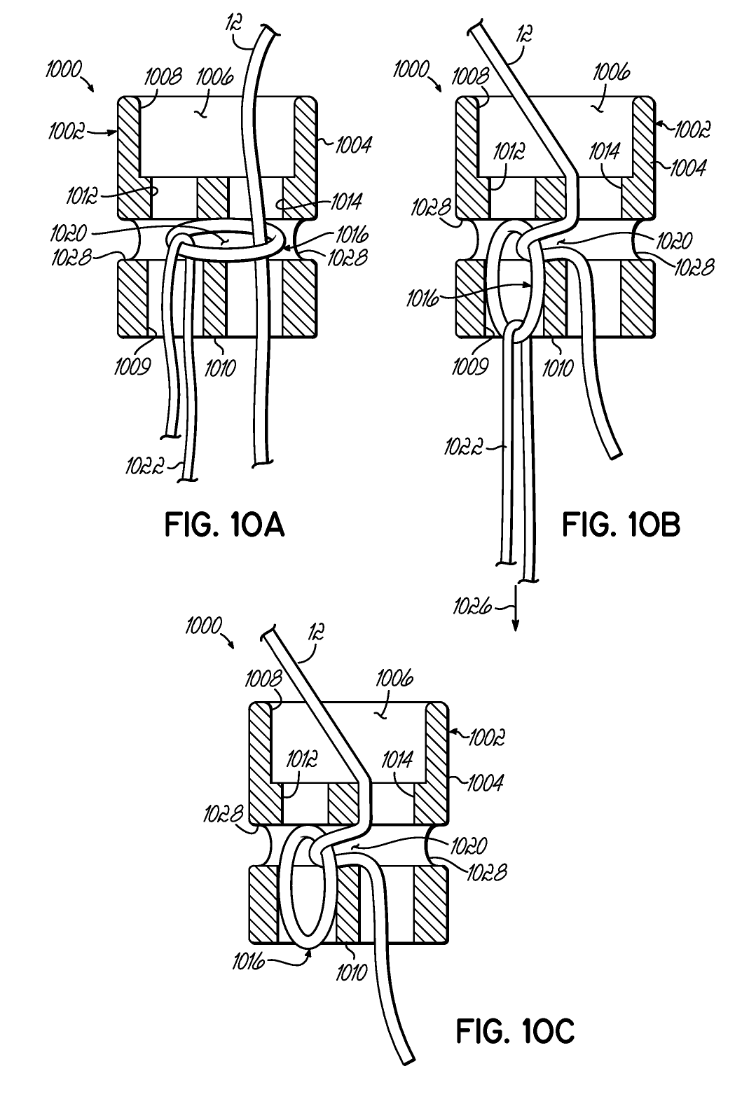

With reference to FIGS. 10A-C and in accordance with an alternative embodiment of the invention, a suture locker 1000 comprises a locker body 1002 with a sidewall 1004 and a passageway or lumen 1006 enclosed by the sidewall 1004. The lumen 1006 extends from a distal aperture 1008 of the locker body 1002 to an intermediate location between the distal aperture 1008 and a bifurcated proximal aperture 1009 of the locker body 1002. A dividing wall 1010 extends from one side of the sidewall 1004 to an opposite side of the sidewall so as to partition or bifurcate a portion of the lumen 1006 to define a pair of smaller channels or lumens 1012, 1014. The lumens 1012, 1014, which originate at the bifurcated proximal aperture 1009, intersect and communicate with the larger lumen 1006.

One or more tensioning members 12 may be threaded through the larger lumen 1006 and one of the smaller lumens, in this particular instance lumen 1014. The tensioning members 12 may also be threaded through the interior of a closed loop 1016 disposed inside the locker body 1002. The dividing wall 1010 is broken by an opening 1020 so that the closed loop 1016, when the tensioning members 12 are free to move axially through the lumens 1006, 1014, is partially disposed in each of the smaller lumens 1012, 1014. A cross bore extending across the lumens 1012, 1014 and through the dividing wall 1010 forms opening 1020. A pulling member (e.g., a pull wire, suture, etc.) 1022 extending through the smaller lumen 1012 is also coupled with the loop 1016. The pulling member 1022 extends distally through the catheter to the physician or healthcare technician.

The tensioning members 12 may be attached by, for example, anchors (not shown) to the intended biological tissue, and one or more plications (not shown) may be formed by pulling the tensioning members 12 generally in the proximal direction 1026. A proximal force applied to the pulling member 1022 withdraws the closed loop 1016 from the smaller lumen 1014 and, as it does so, pulls a looped portion of the tensioning members 12 through the opening 1020 in the dividing wall 1010 and into the other smaller lumen 1012. In order to navigate the angled turn of the tortuous path defined by the opening 1020 and smaller lumen 1012 when the proximal force is applied to the pulling member 1022, the closed loop 1016 should be constructed of a deformable material.

In the activated condition of FIG. 10B, the tensioning members 12 are locked in place or captivated within the tortuous path because of interference developed by the cooperation between the tensioning members 12, the closed loop 1016, the opening 1020, and the lumen 1012. Looped portions of the tensioning members 12 are bunched and contact portions of the dividing wall 1010 that bound the opening 1020. As shown in FIG. 10C, the pulling member 1022 is removed after the tensioning members 12 are secured with the suture locker 1000. The suture locker 1000 secures the tensioning members 12 against movement relative to each other and relative to the suture locker 1000, which prevents relaxation of the plications formed in the tissue using the tensioning members 12.

The suture locker 1000 may be deployed and activated using a catheter assembly similar to catheter assembly 342 described above. In particular, locker body 1002 includes a pair of opposed apertures 1028 adapted to receive a pair of spring arms on the tip of the catheter assembly to couple the suture locker 1000 thereto. When the tensioning members have been suitably tensioned, the pulling member 1022 may be pulled to activate suture locker 1000 as explained above. The suture locker 1000 may then be released from the catheter assembly by releasing the spring arms from apertures 1028. The catheter assembly may then be retracted from the surgical site.

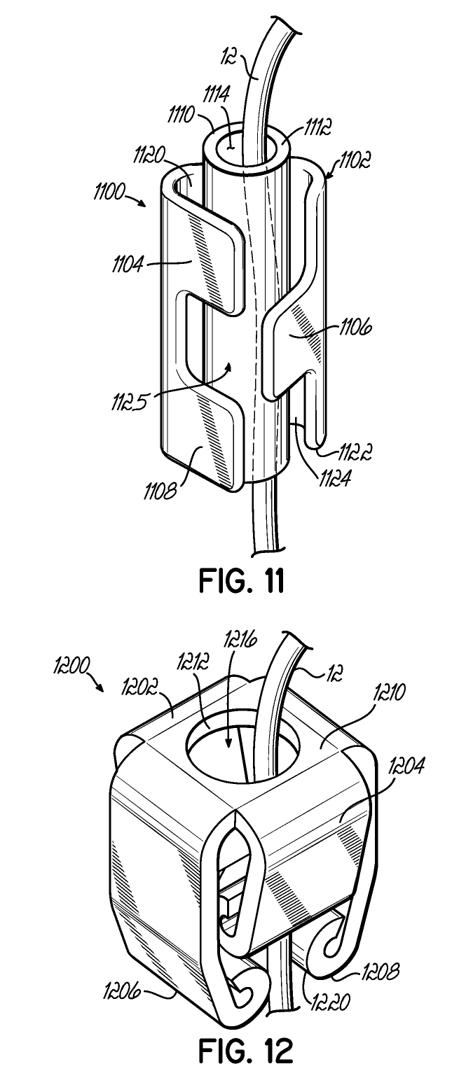

With reference to FIG. 11 and in accordance with an alternative embodiment of the invention, a suture locker 1100 comprises a locker body 1102 having a plurality of interleaved clamping arms 1104, 1106, 1108 that are spaced along a major axis of the locker body 1102. A tubular member 1110 is received in a space circumscribed by the locker body 1102. A thin sidewall 1112 extends between opposite open distal and proximal ends of the tubular member 1110. The sidewall 1112 surrounds a lumen 1114 through which one or more tensioning members 12 are threaded.

The locker body 1102 includes a distal aperture 1120, a proximal aperture 1122, a solid panel 1124 extending between the distal and proximal apertures 1120, 1122, and a gap 1125 extending between the distal and proximal apertures 1120, 1222. The arms 1104, 1106, 1108 are separated by gap 1125, which intersects the distal and proximal edges of the locker body 1102 bordering the apertures 1120, 1122 such that the locker body 1102 is divided along the confronting edges 1120, 1122.

Arms 1104, 1108, which are joined to the solid panel 1124 by smoothly curved transition regions, are angled inwardly from one side edge of the solid panel 1124 and generally overlie the solid panel 1124 at locations near the distal and proximal apertures 1120, 1122, respectively. Arm 1106, which is also joined to the solid panel 1124 by a curved transition region, is angled inwardly from an opposite side edge of the solid panel 1124 and generally overlies the solid panel 1124 at a central location between the distal and proximal apertures 1120, 1122. Arm 1106 is registered with a notch of gap 1125 defined between the arms 1104, 1108 to provide clearance such that, when the arms 1104, 1106, 1108 are moved to clamp the tubular member 1110, arm 1106 fits between arms 1104, 1108.

The locker body 1102 is constructed from a shape memory alloy capable of exhibiting a shape memory effect in which a transformation between phases may be caused by a change in temperature, such the warming experienced after the suture locker 1100 is inserted into a patient's body. The locker body 1102 is provided with a desired configuration in which the arms 1104, 1106, 1108 are open by metallurgical methods understood by persons having ordinary skill in the art. Generally, the locker body 1102 may be formed from a metal alloy that is cold-worked and heat treated to provide the shape memory condition. For example, the metal shape memory alloy may be a nickel titanium (NiTi) alloy tailored to define the temperature at which particular desired shape memory characteristics are obtained. Additional elements may be added to the alloy to affect, for example, the temperature at which particular desired shape memory characteristics are obtained.

In use, the locker body 1102 is conveyed by a catheter assembly to the surgical site in a first condition in which one or more tensioning members 12 are movable through the lumen 1114 of the tubular member 1110. At the surgical site, the tensioning members 12 are pulled in the proximal direction through the suture locker 1100 to form one or more plications in the biological tissue attached to a distal end of the tensioning members 12. The shape memory material of the locker body 1102 changes shape such that the arms 1104, 1106, 1108 move inwardly to pinch, crimp, or otherwise crush the tubular member 1110. When the arms 1104, 1106, 1108 contact the side wall 1112 of the tubular member 1110 and apply a clamping force, the side wall 1112 collapses inwardly to trap and secure the tensioning members 12 between the arms 1104, 1106, 1108 and solid panel 1124. The clamping force prevent axial movement of the tensioning members 12 relative to the apertures 1120, 1122 of tubular member 1110 and relative to each other in either the distal direction or proximal direction.

The suture locker 1100 may be deployed and activated using a catheter assembly having a catheter with a tip adapted to releasably couple with, for example, the proximal end of the locker body 1102. The tensioning members 12 are suitably tensioned prior to the shape memory allow transitioning to the second condition and place the suture locker in the activated condition. The suture locker 1100 may then be released from the catheter assembly and the catheter assembly retracted from the surgical site.

With reference to FIG. 12 and in accordance with an alternative embodiment of the invention, a suture locker 1200 comprises a plurality of spring-biased clamping arms 1202, 1204, 1206, 1208 that project in a proximal direction from peripheral side edges of a central plate 1210. One or more tensioning members 12 may be threaded through a central distal aperture 1212 defined in the central plate 1210 and extend between the arms 1202, 1204, 1206, 1208, which collectively define a passageway 1216 for the tensioning members 12. Two of the arms 1202, 1204 project from opposite side edges of the central plate 1210 so that their corresponding free ends are confronting and separated by a small gap between which the tensioning members 12 are threaded through central aperture 1212. The other two arms 1206, 1208 also project from opposite side edges of the central plate 1210 so that their corresponding free ends are confronting and separated by a small gap between which the tensioning members 12 are threaded. This small gap defines a proximal aperture 1220 for the passageway 1216. Arms 1202, 1204 and arms 1206, 1208 define two axially spaced locations at which the tensioning members 12 may be trapped.

The arms 1202, 1204 and arms 1206, 1208 project in a proximal direction away from the central plate 1210. The tensioning members 12 are free to move in a proximal direction generally away from the aperture 1212 for forming plications in biological tissue. However, bi-directional movement of the tensioning members 12 in the reverse distal direction toward the central plate 1210 is resisted because of the pinching or clamping action of the arms 1202, 1204, 1206, 1208 on the tensioning members 12.

The suture locker 1200 may be deployed and activated using a catheter assembly similar to that described below for suture locker 1300. In particular, the catheter assembly includes a catheter having a tip adapted to releasably couple with, for example, the proximal end of suture locker 1200. The suture locker may include a separating member, similar to separating member 1308, for holding the free ends of arms 1202, 1204, 1206, 1208 apart in a spread condition so that the tensioning members are free to move relative to the suture locker 1200. The separating member has a sufficient rigidity to withstand the clamping force applied by arms 1202, 1204, 1206, 1208 without collapsing so that axial movement of tensioning members 12 is substantially uninhibited. A pulling member may be coupled to the separating member for moving the separating member in the proximal direction. When the tensioning members 12 have been suitably tensioned, the pulling member may be pulled to move the separating member and allow the spring arms 1202, 1204, 1206, 1208 to pinch the tensioning members and place the suture locker in the activated condition. The suture locker may then be released from the catheter assembly and the catheter assembly retracted from the surgical site.

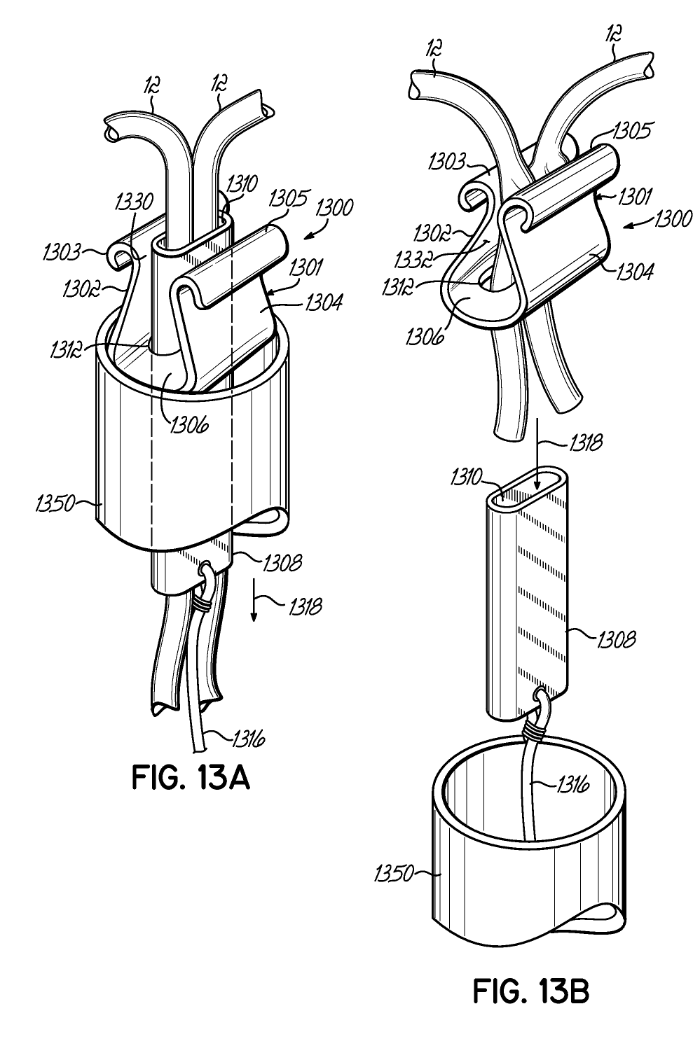

With reference to FIGS. 13A and 13B and in accordance with an alternative embodiment of the invention, a suture locker 1300 comprises a locker body 1301 having a pair of clamping arms 1302, 1304 joined by a curved end 1306. Arms 1302, 1304 project in a distal direction from the curved end 1306. Arm 1302 has a distal free end 1303 and arm 1304 also has a distal free end 1305. The arms 1302, 1304 are arranged such that the distal free ends 1303, 1305 confront each other to define a distal aperture 1330.

A removable separating member 1308 holds the distal free ends 1303, 1305 of the arms 1302, 1304 apart in a spread condition so that one or more tensioning members 12, which are threaded through a passageway or lumen 1310 in the separating member 1308, are free to move relative to the suture locker 1300. The separating member 1308 has a rigidity sufficient to withstand the clamping force applied by the arms 1302, 1304 of the locker body 1301 without collapsing so that axial movement of the tensioning members 12 is substantially uninhibited. The separating member 1308 projects through a proximal aperture 1312 defined in the curved end 1306 of the clip. A pulling member 1316 may be coupled with the separating member 1310 for moving the separating member 1310 in a proximal direction, as indicated by the single-headed arrows 1318, relative to the clip and toward the physician or healthcare technician.

The curved end 1306 of the locker body 1301 is constructed such that the arms 1302, 1304 are resilient and, when the separating member 1310 is removed as shown in FIG. 13B to deploy and activate the suture locker 1300, the arms 1302, 1304 are free to cantilever relative to the curved end 1306 and generally move toward each other for closing a passageway 1332 between the proximal and distal apertures 1312, 1330. After one or more plications are formed in the tissue to which the distal ends of the tensioning members 12 may be attached by, for example, moving the tensioning members 12 in the proximal direction 1318 through the lumen 1310 in the separating member 1310, the tensioning members 12 are captured or pinched between the confronting free ends 1303, 1305 of arms 1302, 1304. The applied pinching force secures the tensioning members 12 in passageway 1332 against further movement relative to the locker body 1301 of the suture locker 1300 and relative to each other in either the distal or proximal directions.

The suture locker 1300 may be deployed and activated using a catheter assembly having a catheter 1350 with a tip adapted to releasably couple with, for example, the proximal end of suture locker 1300. When the tensioning members 12 have been suitably tensioned, the pulling member 1316 may be pulled to remove separating member 1308 and activate suture locker 1300. The suture locker 1300 may then be released from the catheter assembly and the catheter assembly retracted from the surgical site.

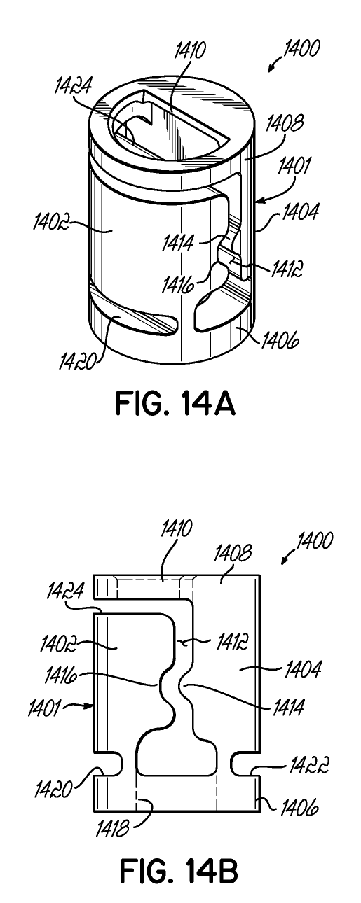

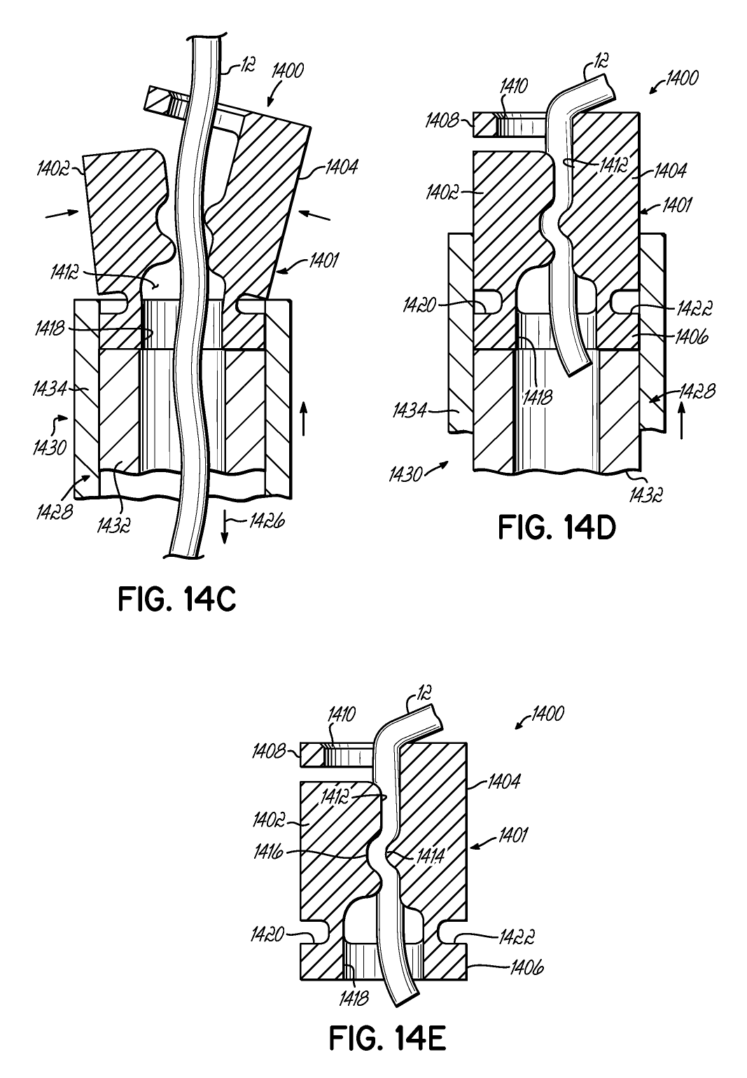

With reference to FIGS. 14A and 14B and in accordance with an alternative embodiment of the invention, a suture locker 1400 comprises a locker body 1401 having a pair of contoured clamping arms 1402, 1404 and a proximal ring 1406 joining the clamping arms 1402, 1404. A disk 1408 of locker body 1401 having a D-shaped opening 1410 depends approximately perpendicular to the free, distal end of clamping arm 1404. The curved portion of the D-shaped opening or aperture 1410 projects over a distal end 1424 of clamping arm 1402 and the linear portion of the D-shaped aperture 1410 is located near the axial centerline of the locker body 1401.

The clamping arms 1402, 1404 are separated by a gap or passageway 1412 extending between the disk 1408 and the proximal ring 1406 and aligned generally in a proximal-distal direction. Clamping arm 1404 includes a ridge 1414 aligned transverse to the proximal-distal direction. Clamping arm 1402 includes a groove 1416 that is also aligned transverse to the proximal-distal direction and shaped to receive the ridge 1414 of clamping arm 1404 when the suture locker 1400 is activated. Near the respective attachment points to the proximal ring 1406, the clamping arms 1402, 1404 are weakened by notches 1420, 1422, which promotes the ability to deflect the clamping arms 1402, 1404 relative to the proximal ring 1406 and provides locations for grasping the suture locker 1400. The gap 1412 is also enlarged near the notches 1420, 1422. The proximal ring 1406 further includes a central opening or aperture 1418 through which one or more tensioning members 12 may be threaded. The tensioning members 12 may also be threaded through the D-shaped aperture 1410 in disk 1408.

In use and with reference to FIGS. 14C-E, the tensioning members 12 are threaded through the apertures 1410, 1418 and, thereby, disposed in passageway 1412 between the clamping arms 1402, 1404 of the locker body 1401. The clamping arms 1402, 1404 are spread so that the passageway 1412 is sufficiently wide to permit free axial movement of the tensioning members 12. One or more plications may be formed in the tissue to which the distal ends of the tensioning members 12 are attached by, for example, moving the tensioning members 12 in a proximal direction 1426. After forming the plications, the suture locker 1400 is pulled into the open end of a tubular member 1428. Contact between the clamping arms 1402, 1404 and the inner surface of the lumen of the tubular member 1428 prompts the clamping arms 1402, 1404 of the locker body 1401 to move toward each other, which narrows the passageway 1412. The clamping arms 1402, 1404 contact the tensioning members 12 and apply a compressive force that captivates the tensioning members 12. The routing of the tensioning members 12 between the ridge 1414 and groove 1416 aids in preventing relative movement of the tensioning members 12 after the suture locker 1400 is activated.

The suture locker 1400 may be deployed and activated using a catheter assembly 1430. For example, tubular member 1428 may form a part of catheter assembly 1430. In particular, catheter assembly 1430 may include an inner catheter 1432 that may be releasably coupled to, for example, the proximal end of suture locker 1400 and an outer catheter 1432 movable relative to the inner catheter 1432. During deployment, the suture locker 1400 is in the latent condition so as to allow the tensioning members 12 to pass therethrough relatively uninhibited. When the tensioning members have been sufficiently tensioned, the outer catheter 1434 may move relative to the inner catheter 1432 (or vice versa) so as to activate the suture locker 1400 as described above. The suture locker 1400 may then be released from catheter assembly 1430 and the catheter assembly retracted from the surgical site.

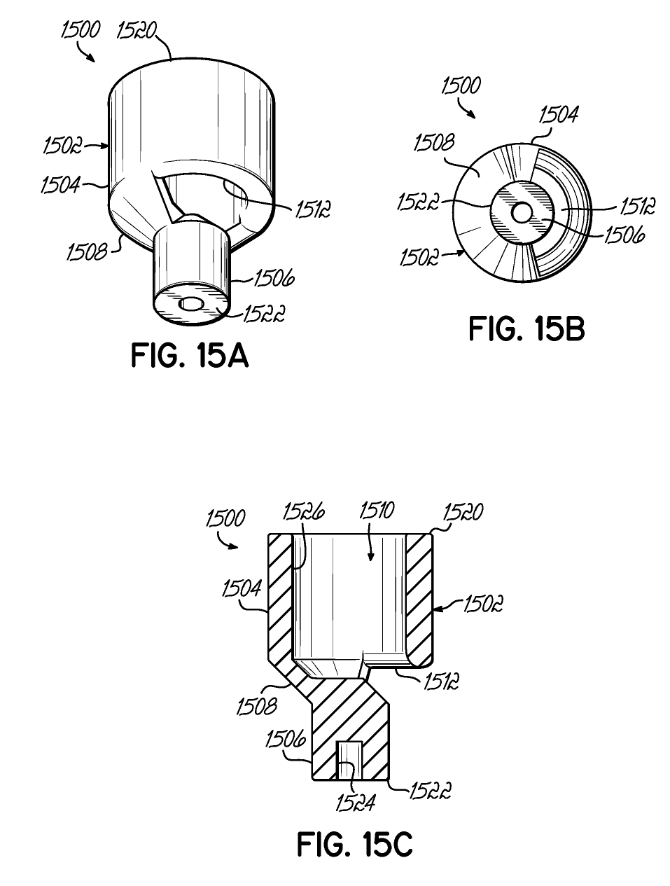

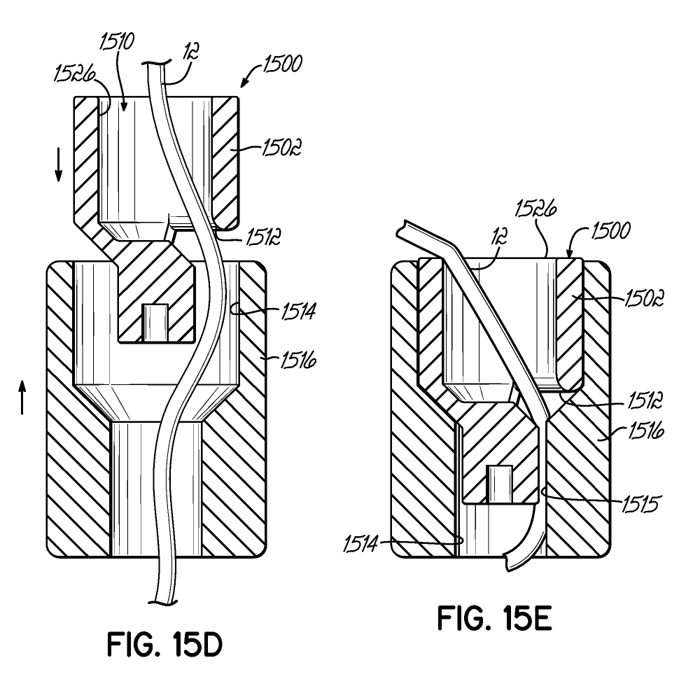

With reference to FIGS. 15A-C and in accordance with an alternative embodiment of the invention, a suture locker 1500 comprises a locker body 1502 with a tubular distal section 1504 and a tubular proximal section 1506 of smaller outer diameter joined by an tapered section 1508. Distal section 1504 encloses a passageway or lumen 1510 having an inner diameter greater than the outer diameter of proximal section 1506.

Penetrating through the tapered section 1508 is a proximal opening or aperture 1512 that communicates with the lumen 1510. The proximal aperture 1512 extends circumferentially about the locker body 1502 and has a radial dimension relative to the axis of symmetry of the locker body 1502. A distal aperture 1526 terminates the lumen 1510 near the surgical site. The diametric difference between the distal and proximal sections 1504, 1506 of the locker body 1502 provide clearance for the proximal aperture 1512, which is offset from the axis of symmetry of the locker body 1502. The locker body 1502 extends between a distal end 1520 and a proximal end 1522. The proximal section 1506 includes structure 1524 disposed near the proximal end 1522 that is used to grip the suture locker 1500, such as with a pulling member.

With reference to FIGS. 15D and 15E, one or more tensioning members 12 may be threaded through the lumen 1510 and the apertures 1512, 1526. The distal end of each tensioning member 12 may be attached to biological tissue. Movement of the tensioning members 12 in a proximal direction applies a force to the tensioning members that may prompt the formation of one or more plications. After the plications are formed, the suture locker 1500 may be moved into a complementary-shaped lumen 1514 inside a tubular body 1516 to form an assembly. The tensioning members 12 are captivated between the exterior surface of the suture locker 1500 and a portion 1515 of the inner surface of the tubular body 1516 bordering the lumen 1514. This secures the tensioning members 12 against movement relative to each other and relative to the suture locker 1500, which prevents the plications from relaxing. The space inside the tubular body 1516 above the inner surface portion 1515 is contoured complementary to the shape of the distal and tapered sections 1504, 1508 to provide clearance for moving the suture locker 1500 into the tubular body 1516.

The suture locker may be deployed and activated using a catheter assembly with a tip adapted to releasably couple with, for example, the proximal end of tubular body 1516. The catheter assembly may further include a pulling member operatively coupled to suture locker 1500, such as at 1524. When the tensioning members 12 have been suitably tensioned, the pulling member may be pulled such that the suture locker 1500 is pulled inside tubular body 1516 to place the suture locker in the activated condition. The tubular body 1516 may be released from the catheter assembly and the catheter assembly retracted from the surgical site.

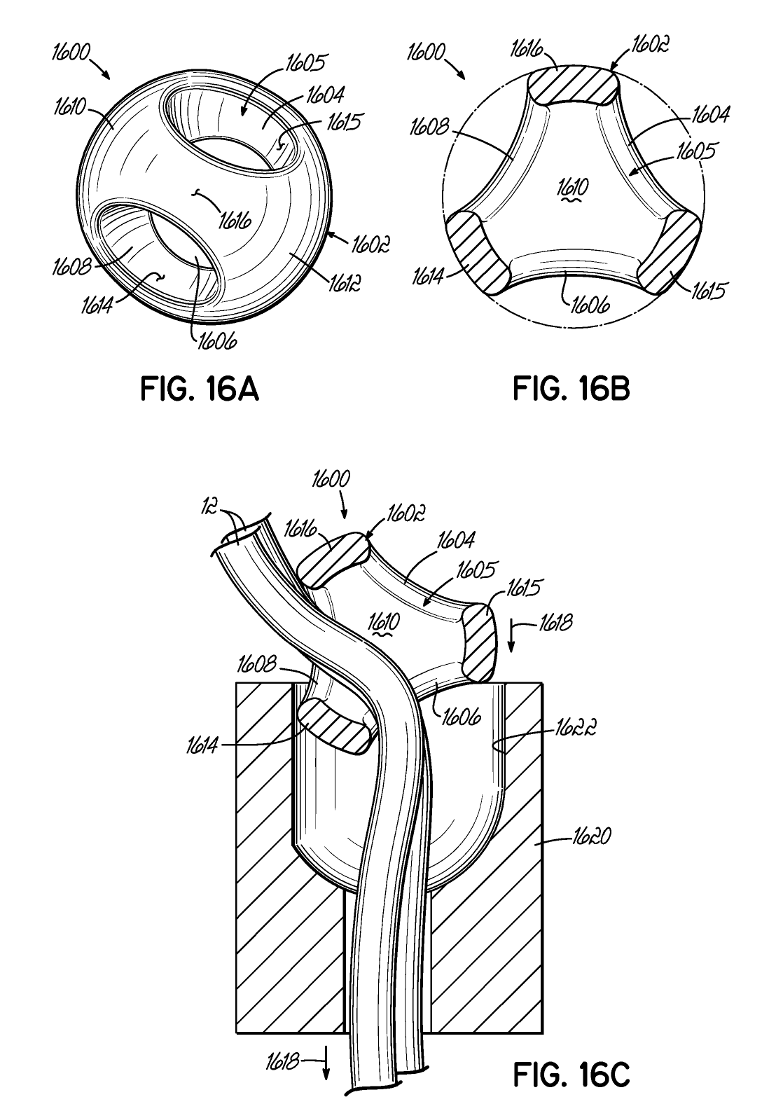

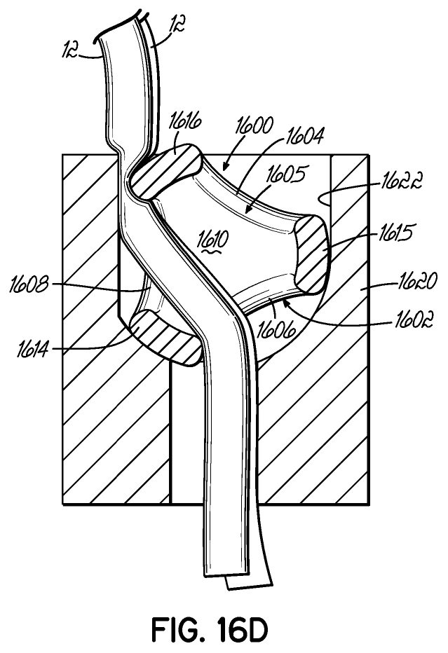

With reference to FIGS. 16A and 16B and in accordance with an alternative embodiment of the invention, a suture locker 1600 comprises a generally spherical locker body 1602 broken by a plurality of passageways 1604, 1606, 1608 that communicate from the exterior of the suture locker 1600. A central cavity 1605 is defined inside the locker body 1602 by the intersection of the passageways 1604, 1606, 1608. The locker body 1602 includes a plurality of concave hubs 1610, 1612 and a plurality of curved spokes 1614, 1615, 1616 that connect the concave hubs 1610, 1612. The hubs 1610, 1612 are formed at the intersection of the spokes 1614, 1615, 1616.

One or more tensioning members 12 may be threaded through two of the passageways 1604, 1606, as shown in FIG. 16C. After the distal ends of the tensioning members 12 are fastened to the tissue, plications may be created in the biological tissue by moving the tensioning members 12 through the suture locker 1600 in a proximal direction. With the tensioning members 12 held in tension, the suture locker 1600 is pulled in a proximal direction 1618 into a tubular member 1620. As depicted in FIG. 16D, the tensioning members 12 are pinched between the exterior of the locker body 1602 and an interior wall 1622 of the tubular member 1620, which captivates the tensioning members 12. As a result, the plications do not relax when the proximally-directed force applied to the tensioning members 12 is released.

The suture locker 1600 may be deployed and activated using a catheter assembly with a tip adapted to releasably couple with, for example, the proximal end of tubular member 1620. The catheter assembly may further include a pulling member operatively coupled to suture locker 1600. When the tensioning members 12 have been suitably tensioned, the pulling member may be pulled such that the suture locker 1600 is pulled inside tubular member 1620 to place the suture locker in the activated condition. The tubular member 1620 may be released from the catheter assembly and the catheter assembly retracted from the surgical site.

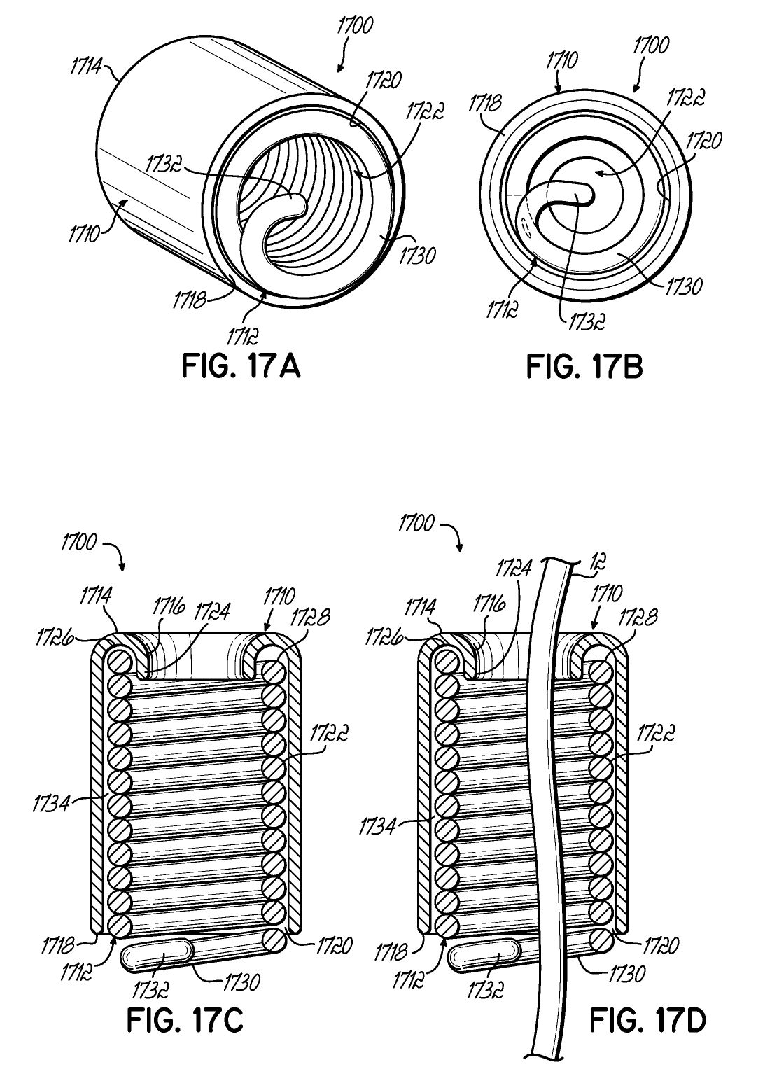

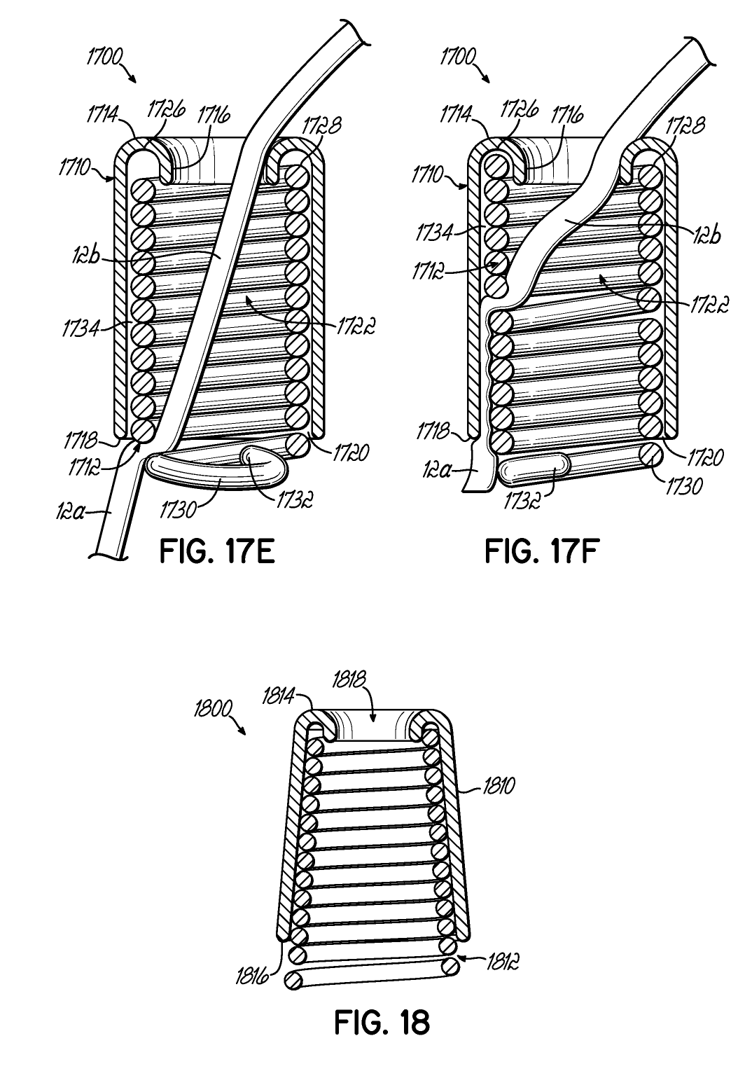

With reference to FIGS. 17A-17F and in accordance with an alternative embodiment of the invention, a suture locker 1700 includes an outer tubular locker body 1710 and an inner expandable member, such as a coil spring 1712, positioned in locker body 1710 for securing tensioning members 12. The locker body 1710 includes a distal end 1714 having an aperture 1716, a proximal end 1718 having aperture 1720 and a passageway 1722 extending between distal and proximal ends 1716, 1720 and through locker body 1710. Distal end 1714 includes an inwardly directed U-shaped flange 1724 that defines a proximally-facing interior cavity 1726. The locker body 1710 has an inside diameter suitable to receive coil spring 1712 in passageway 1722. The coil spring 1712 has a distal end 1728 that is received in cavity 1726 and secured to locker body 1710 at distal end 1714. For instance, coil spring 1712 may be welded to U-shaped flange 1724. A proximal end 1730 of spring 1712 extends slightly beyond the proximal end 1718 of locker body 1710. The proximal end 1730 includes a tail portion 1732 that projects radially inward and downward so as to create a slight separation between the tail 1732 and the coil immediately adjacent the tail 1732. The coil spring 1712 is closely received in locker body 1710 such that there is a small gap 1734 between the outer surface of the spring 1712 and the inner surface of passageway 1722.

Operation of the suture locker 1700 is illustrated in FIGS. 17D-17F. As shown in FIG. 17D, one or more tensioning members 12 may be threaded through aperture 1716, passageway 1722 and aperture 1718 of locker body 1710 so as to be located on the interior side of the coil spring 1712. In this latent condition, the tensioning members 12 may freely move relative to the suture locker 1700 through passageway 1722 in the proximal-distal direction. As shown in FIG. 17E, to activate suture locker 1700, the tensioning members 12 may be moved between the tail 1732 and the adjacent coil 1736 and rotated or wound along the coils of the spring 1712. This moves a proximal portion 12a of the tensioning members 12 external to coil spring 1712 while a distal portion 12b of tension members 12 remain internal to coil spring 1712. As the tensioning members 12 are wound along the coils, the spring 1712 may be stretched or expanded along the axis of the coil spring 1712 so that the proximal portion 12a of tensioning members 12 remain outside the proximal end 1718 of locker body 1710. Alternatively, the spring 1712 may be stretched or expanded using a pulling member.

After several revolutions of winding the tensioning members 12 along the coils of spring 1712, the tensioning members 12 may be released. When the tensioning members 12 are released, the coil spring 1712 compresses back toward its unstretched state causing the proximal portion 12a of tensioning members 12 external to spring 1712 to be pulled up within the locker body 1710 through aperture 1720 in proximal end 1718. As shown in FIG. 17F, at least part of proximal portion 12a of tensioning members 12 is positioned in gap 1734 and captured between the outer surface of coil spring 1712 and passageway 1722. The tensioning members 12 are also captured between two adjacent coils of spring 1712 at the location where the tensioning members 12 go from being internal to coil spring 1712 to being external coil spring 1712. In this activated condition, the tensioning members 12 are no longer free to move relative to the locker body 1710 nor are the tensioning members 12 free to move relative to each other.

The suture locker 1700 may be deployed and activated using a catheter assembly having a catheter with a tip adapted to releasably couple with, for example, the proximal end of locker body 1710. The catheter assembly may further include a pulling member operatively coupled to the coil spring 1712, such as at tail 1732, so as to maintain the suture locker 1700 in the latent condition during deployment. When the tensioning members have been suitably tensioned, the pulling member may be released so as to allow the spring 1712 to contract to pinch the tensioning members 12 and place the suture locker 1700 in the activated condition. The suture locker may be released from the catheter assembly and the catheter assembly retracted from the surgical site.

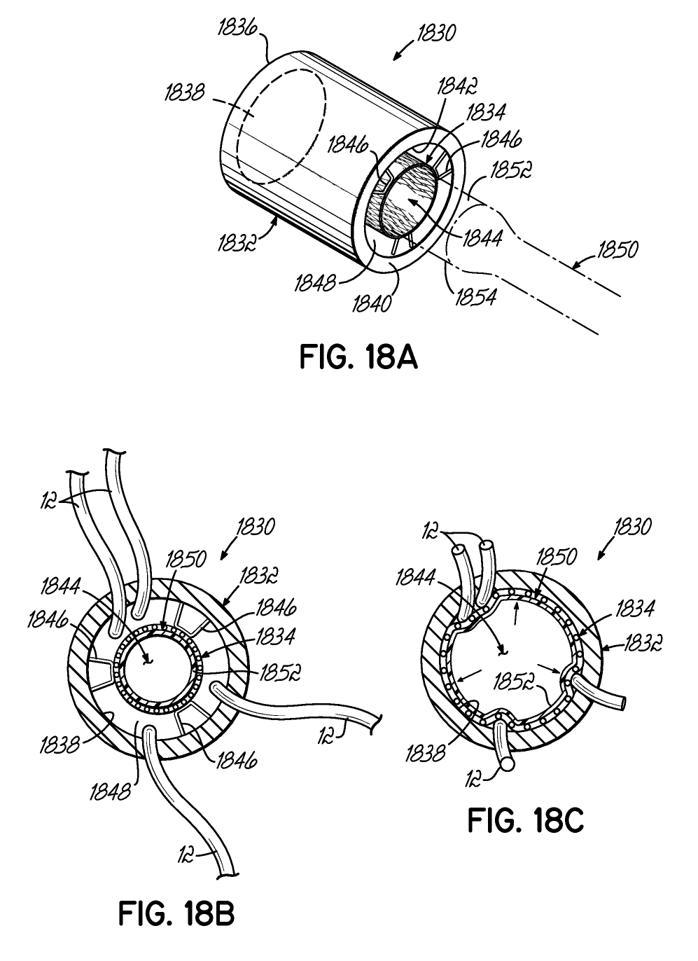

With reference to FIG. 18 and in accordance with an alternate embodiment of the invention shown in FIGS. 17A-17F and described above, a suture locker 1800 includes a locker body 1810 and expandable member, such as a spring 1812 having a tapered or frustoconical configuration with the diameter of the locker body 1810 and spring 1820 increasing from a distal end 1814 to a proximal end 1816 of locker body 1810. In this way, as the tensioning members 12 are wound along the coils of spring 1812 and the spring 1812 stretches so as to bring a small diameter portion of the spring toward the proximal end 1816 of locker body 1810, a gap is formed between the outer surface of the spring 1812 and the inner surface of the passageway 1818 defined through locker body 1810. As described above, when the tension in the tensioning members 12 is released, the tensioning members 12 are pulled up into the locker body 1810 and compressed between the spring 1812 and the passageway 1818 and between adjacent coils of spring 1812. Suture locker 1800 may be delivered and deployed in a manner similar to that described above for locker 1700.

With reference to FIGS. 18A-18C and in accordance with an alternative embodiment of the invention similar to that shown in FIGS. 17A-17F, a suture locker 1830 includes an outer tubular locker body 1832 and an inner expandable member, such as an expandable mesh tubular member 1834 (e.g., a stent-like member), positioned in the locker body 1832 for securing tensioning members 12. The outer tubular locker body 1832 may be a rigid tubular body made of a suitable biocompatible material, or may include a stent-like tubular member similar to expandable member 134, a coil spring, or a hollow braided member. The locker body 1832 includes a distal end 1836 having an aperture 1838, a proximal end 1840 having an aperture 1842, and a passageway 1844 extending between distal and proximal ends 1836, 1840 and through the locker body 1832. The expandable tubular member 1834 is positioned in the passageway 1844 and is coupled to the locker body 1832 via one or more flexible connectors 1846, which may be located at one or both of the distal and proximal ends 1836, 1840, or alternatively, may extend along the locker body 1832 and expandable tubular member 1834 between the distal and proximal ends 1836, 1840. The expandable tubular member 1834 is expandable between a compressed position and an expanded position in, for example, a generally radial direction.

Operation of the suture locker 1830 is illustrated in FIGS. 18B and 18C. As shown in FIG. 18B, one or more tensioning members may be threaded through aperture 1838, passageway 1844 and aperture 1842 of locker body 1832 so as to be located in a gap 1848 defined between the locker body 1832 and the expandable tubular member 1834 when the stent is in the compressed position. In this latent condition, the tensioning members 12 may freely move relative to the suture locker 1830 through passageway 1844 in the proximal-distal direction. As shown in FIGS. 18A and 18C, the suture locker 1830 may be activated using a balloon catheter 1850 having an expandable balloon 1852 at its tip, as is generally known in the art. In the compressed position, the expandable tubular member 1834 is essentially tightly wrapped around an outer surface 1854 of the expandable balloon 1852 when in a non-expanded state. Once the tensioning members 12, the distal ends of which may be attached to tissue, such as with an anchor, have been tensioned, the balloon catheter 1850 may be actuated so as to expand the balloon 1852 and thus radially expand the expandable tubular member 1834 (FIG. 18D). In this activated condition, the stent 1834 applies a radially outward bias such that the tensioning members 12 are captured between the stent 1834 and the locker body 1832 so that the tensioning members 12 may not move relative to the suture locker 1830 or relative to each other.

The suture locker 1830 may be deployed and activated using a catheter assembly having an outer catheter which may be releasably coupled to the locker body 1832 and the inner balloon catheter 1850 used to expand the expandable tubular member 1834. When the tensioning members 12 have been suitably tensioned, the balloon 1852 is expanded to expand the expandable tubular member 1834 and place the suture locker in the activated condition. The balloon 1852 may then be deflated and the suture locker 1830 released from the catheter assembly. The catheter assembly may then be retracted from the surgical site.

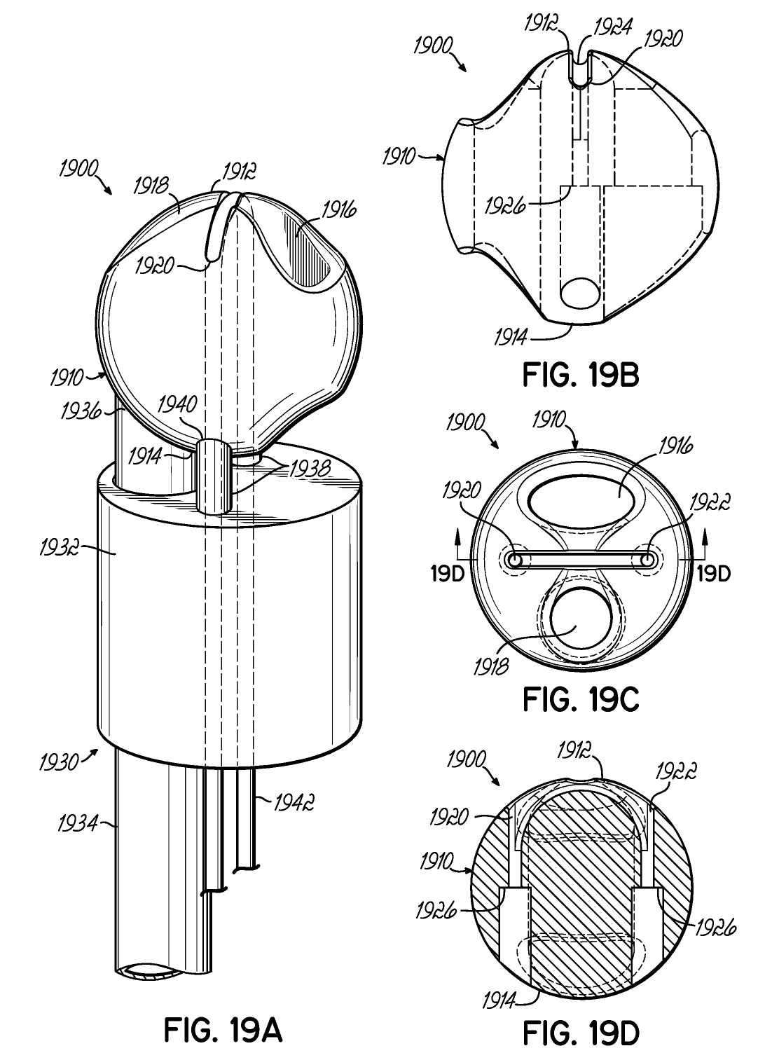

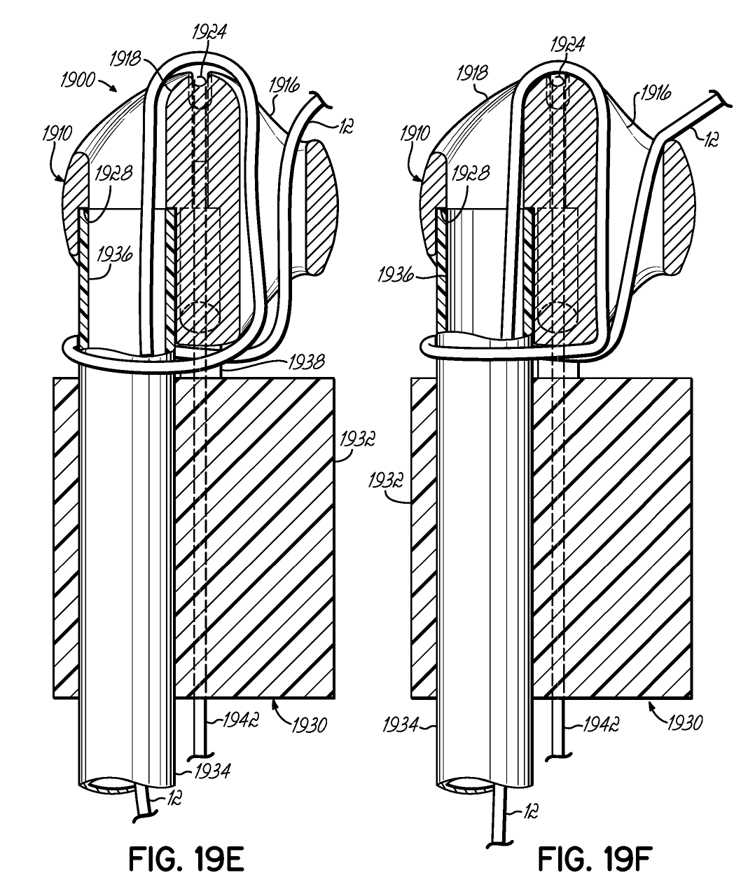

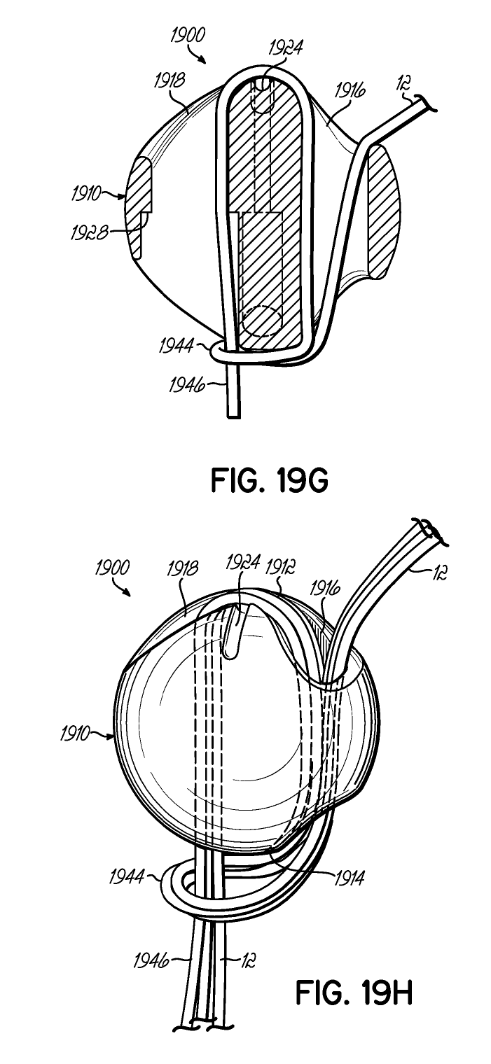

With reference to FIGS. 19A-H and in accordance with an alternative embodiment of the invention, a suture locker 1900 includes a generally spherical locker body 1910 having a distal end 1912, a proximal end 1914, and first and second spaced apart bores 1916, 1918, respectively, formed therein that extend substantially in the proximal-distal direction. First bore 1916 has a generally elliptical cross section and second bore 1918 has a generally cylindrical cross section. Suture locker 1900 further includes third and fourth spaced-apart bores 1920, 1922 that are ninety degrees offset from the first and second bores 1916, 1918 and likewise extend substantially in the proximal-distal direction. A groove 1924 extends across the distal end 1912 of the locker body 1910 between the third and fourth bores 1920, 1922. As shown in FIGS. 19B and 19D, bores 1920, 1922 have a proximal portion with a diameter that is greater than the diameter of bores 1920, 1922 along a distal portion so as to define an interior shoulder 1926. As shown in FIG. 9E, bore 1918 has a similar construction so as to define interior shoulder 1928 therein.

FIG. 19A illustrates catheter assembly 1930 for delivery of the suture locker 1900 in a catheter-based system. As shown, the suture locker 1900 is located on the distal tip of a delivery catheter 1932. The deliver catheter 1932 includes a hypo tube 1934 having one end outside the body (not shown) and the other end 1936 positioned in the proximal end of bore 1918. As shown in FIG. 19E, the distal end 1936 of hypo tube 1934 engages shoulder 1928. The delivery catheter 1932 further includes a pair of support tubes 1938 each having a proximal end coupled to the tip of delivery catheter 1932 and a distal end 1940 positioned in the proximal end of bores 1920, 1922 so as to engage shoulders 1926. As shown in FIG. 19A, a pulling member 1942 is threaded up through the catheter 1932, through one of the support tubes 1938, through one of bores 1920, 1922, across groove 1924 and back down through the other bore 1920, 1922, support tube 1938, and back through catheter 1932. The pulling member 1942 selectively retains the suture locker 1900 on the tip of catheter 1932. When the suture locker 1900 is ready to be released, the pulling member 1942 is removed.

With reference to FIGS. 19E-19H, the distal end of one or more tensioning members 12 may be attached to tissue, such as with an anchor. Movement of the tensioning members 12 in the proximal direction applies a force to the tensioning members 12 that may prompt the formation of one or more plications in the tissue. To maintain the plications in the tissue, the movement of the tensioning members relative to each other is restrained. To this end, the tensioning members 12 are threaded through bore 1916 in the proximal direction, wrapped around hypo tube 1934, threaded back through bore 1916 in the distal direction, and threaded through bore 1918 so as to extend through hypo tube 1934 and outside the body for manipulation. Suture locker 1900 may be suitably positioned with respect to the tissue and the support tubes 1938 and hypo tube 1934 removed. As illustrated in FIGS. 19G and 19H, the tensioning members 12 form a loop 1944 through which a distal portion 1946 of tensioning members 12 extends. When the tensioning members 12 are tightened, the loop 1944 and distal portion 1946 of tensioning members 12 are pulled into the proximal end of bore 1916. Bore 1916, however, is sized to prevent the passage of the loop 1944 and distal portion 1946 therethrough. Thus, the suture locker 1900 is activated and movement of the tensioning members relative to the suture locker 1900 and relative to each other is prevented.

As used herein, the term “distal” refers to a direction or location near the procedure site and remote from the physician or healthcare technician, and the term “proximal” refers to a direction or location closer to the physician or healthcare technician. Although some of the figures herein may depict a single tensioning member 12, it is understood that one or more tensioning members 12 may be captured by the suture lockers. The dimensions of the tensioning members 12 are understood to be diagrammatic.

While the invention has been illustrated by a description of various illustrative embodiments and while these embodiments have been described in some detail, it is not the intention of the Applicants to restrict or in any way limit the scope of the appended claims to such detail. Additional advantages and modifications will readily appear to those skilled in the art. The various features of the invention may be used alone or in numerous combinations depending on the needs and preferences of the user.

What is claimed is:

1. A locker for securing one or more tensioning members, comprising:
a locker body; and
a means coupled to said locker body for securing the tensioning members, said means having a latent condition wherein the tensioning members are movable with respect to said locker body, and an activated condition wherein the tensioning members are prevented from moving relative to said locker body.

2. A locker for securing one or more tensioning members, comprising:
a locker body having a first aperture, a second aperture, and a passageway extending between said first and second apertures, the tensioning members threaded through said passageway between said first and second apertures; and
a pin coupled to said locker body so as to traverse said passageway, said pin movable in a direction generally between said first and second apertures, and said pin moveable between a first position within said passageway in which the tensioning members are movable relative to said locker body and a second position within said passageway in which the tensioning members are substantially secured against movement relative to said locker body.

3. The locker of claim 2, wherein the tensioning members are secured against movement relative to said locker body through frictional engagement with said pin.

4. The locker of claim 2, further comprising:
a blocking element projecting into said passageway and configured to prevent movement of said pin from the second position and toward the first position.

5. The locker of claim 4, wherein said blocking element is further configured to permit movement of said pin from the first position and toward the second position.

6. The locker of claim 4, wherein said blocking element includes a resilient portion.

7. The locker of claim 4, wherein said blocking element includes a spring clip.

8. The locker of claim 4, wherein said blocking element includes a biasing element that resiliently yields to permit said pin to move from the first position to the second position and that opposes movement of said pin from the second position to the first position.

9. The locker of claim 2, further comprising:
a pair of opposed slots formed in the locker body, said pin being engaged with said slot so that said pin moves along said slot between the first and second positions.

10. The locker of claim 9, wherein each slot includes a first segment and a second segment communicating with said first segment, said first and second segments configured to form an acute angle therebetween.

11. The locker of claim 9, wherein said locker body includes an axis, said opposed slots forming an acute angle with respect to said axis.

12. The locker of claim 2, further comprising:
a biasing member projecting into said passageway and engaging said pin, said biasing member configured to bias said pin toward the second position.

13. The locker of claim 12, wherein said biasing member includes one of a superelastic material, a shape memory material, or an elastomeric material.

14. A locker for securing one or more tensioning members, comprising:
a locker body having a first aperture, a second aperture, and a passageway extending between said first and second apertures; and
a cam member positioned within said passageway and movable between a latent condition and an activated condition,
wherein the tensioning members are movable relative to said locker body when said cam member is in the latent condition, and the tensioning members are prevented from moving relative to said locker body when said cam member is in the activated condition.

15. The locker of claim 14, wherein said cam member includes a plurality of teeth for engaging the tensioning members when in the activated condition.

16. The locker of claim 14, wherein the tensioning members are secured against movement relative to said locker body through frictional engagement with said cam member.

17. The locker of claim 14, further comprising:
a biasing member coupled to said cam member for biasing said cam member toward the activated condition.

18. A locker for securing one or more tensioning members, comprising:
a locker body having a first aperture, a second aperture, and a passageway extending between said first and second apertures;
a pair of opposed slots formed in said locker body;
a first pin coupled to said locker body so as to traverse said passageway and movable along said slots; and
a second pin coupled to said locker body so as to traverse said passageway, said second pin capable of being axially spaced relative to said first pin, said first and second pins defining a latent condition and an activated condition,
wherein the tensioning members are movable relative to said locker body when said first and second pins are in the latent condition, and the tensioning members are prevented from moving relative to said locker body when said first and second pins are in the activated condition.

19. The locker of claim 18, wherein said second pin is fixed relative to said slots.

20. The locker of claim 19, wherein said first pin is movable toward said second pin so as to compress the tensioning members therebetween when in the activated condition.

21. The locker of claim 18, wherein said second pin is movable along said slots.

22. The locker of claim 21, wherein said first and second pins are movable toward each other so as to compress the tensioning members therebetween when in the activated condition.

23. A locker for securing one or more tensioning members, comprising:
a locker body having a first aperture, a second aperture, and a passageway extending between said first and second apertures; and
at least one spring arm positioned within said passageway and movable between a latent condition and an activated condition,
wherein the tensioning members are movable relative to said locker body when said at least one spring arm is in the latent condition, and the tensioning members are prevented from moving relative to said locker body when said at least one spring arm is in the activated condition.

24. The locker of claim 23, wherein said at least one spring arm is biased toward the activated condition.

25. The locker of claim 23, wherein said locker is configured as a one-way locker, wherein said at least one spring arm resiliently yields to permit movement of the tensioning members relative to said locker body in a first direction and opposes movement of the tensioning members relative to said locker body in a second direction opposite the first direction.

26. The locker of claim 23 having a pair of spring arms.

27. The locker of claim 26, wherein said spring arms are axially spaced along said locker body.

28. The locker of claim 26, wherein said pair of spring arms are positioned on opposite sides of the tensioning members so as to capture the tensioning members therebetween when in the activated condition.

29. The locker of claim 23, wherein said passageway includes a generally circular aperture, said spring arm including a ring portion positioned within said circular aperture and adapted to cooperate with said aperture to capture the tensioning members.

30. A locker for securing one or more tensioning members, comprising:
a locker body having at least one passageway, the tensioning members threaded through said at least one passageway, said locker body having a first condition in which the tensioning members are movable relative to said locker body and a second condition in which the tensioning members are captivated within said at least one passageway so as to be secured against movement relative to said locker body.

31. The locker of claim 30, wherein said locker body includes at least two passageways communicating with each other, the locker further comprising:
a pulling member extending into said passageways and coupled with the tensioning members, said pulling member moveable relative to said locker body for moving the tensioning members to generate an interference fit that provides the second condition.

32. The locker of claim 30, further comprising:
a tubular member enclosing a lumen, wherein said locker body is positioned inside said lumen of said tubular member to place said locker body in the second condition.

33. The locker of claim 30, wherein said locker body comprises a first tubular section and a second tubular section having a cross dimension less than the first tubular section, and the tubular member includes a first lumen portion and a second lumen portion, the first lumen portion adapted to receive the first tubular section therein and the second lumen portion adapted to receive the second tubular section therein in a friction fit.

34. The locker of claim 30, wherein said locker body comprises a spherical body including at least two passageways formed therein that intersect each other, the tensioning members entering one of the passageways and exiting from another of the passageways, and the tubular member including a first lumen portion adapted to receive the spherical body therein in a friction fit.

35. A locker for securing one or more tensioning members, comprising:
a locker body having a first aperture, a second aperture, a passageway extending between said first and second apertures, and a plurality of clamping arms separated by said passageway, said clamping arms moveable relative to each other between a first position in which the tensioning members are movable relative to said locker body and a second position in which the tensioning members are clamped between said clamping arms to secure the tensioning members against movement relative to said locker body.

36. The locker of claim 35, wherein said locker body comprises a shape memory alloy that is capable of exhibiting a shape memory effect that provides a transition from said first position to said second position.

37. The locker of claim 35, further comprising:
a collapsible tubular member defining a lumen and positioned in said passageway, the tensioning members being disposed through said lumen of said tubular member, said clamping arms collapsing said tubular member about the tensioning members when said clamping arms are moved to the second position.

38. The locker of claim 35, wherein said clamping arms are resiliently biased relative to each other in a direction to move from the first position to the second position.

39. The locker of claim 35, further comprising:
a separating member disposed between said clamping arms to separate said clamping arms in the first position and removable from between said clamping arms to release said clamping arms for biased movement from the first position to the second position.

40. The locker of claim 35, wherein the clamping arms include a first pair of opposed clamping arms for capturing the tensioning members therebetween when in the second position, and a second pair of opposed clamping arms axially spaced from said first pair of clamping arms for capturing the tension members therebetween when in the second position.

41. A locker for securing one or more tensioning members, comprising:
a rigid locker body having a first aperture, a second aperture, and a passageway extending between said first and second apertures; and
an expandable member coupled to said locker body and disposed in said passageway, said expandable member movable between a latent condition and an activated condition,
wherein the tensioning members are movable relative to said locker body when said expandable member is in the latent condition, and the tensioning members are prevented from moving relative to said locker body when said expandable member is in the activated condition.

42. The locker of claim 41, wherein said expandable member comprises a coil spring generally aligned along said passageway.

43. The locker of claim 42, wherein said coil spring is biased toward the expanded position.

44. The locker of claim 42, wherein the coil spring is elongated when in the latent condition.

45. The locker of claim 41, wherein said expandable member comprises an expandable tubular member.

46. The locker of claim 45, wherein the expanded tubular member is expanded when in the latent condition.

47. The locker of claim 41, wherein the locker body has a tapered configuration.

48. The locker of claim 47, wherein the expandable member has a tapered configuration.

49. The locker of claim 41, wherein the expandable member is expandable in an axial direction to define the latent and activated conditions.

50. The locker of claim 41, wherein the expandable member is expandable in a radial direction to define the latent and activated conditions.

51. An apparatus for securing one or more tensioning members at a surgical site within the body of a patient, comprising:
a catheter assembly having at least a first catheter with a tip, said catheter assembly capable of being disposed in the vasculature of the patient such that said tip is proximate the surgical site; and
a locker releasably coupled to said tip for securing the tensioning members at the surgical site, the locker comprising:
a) a locker body; and
b) a means coupled to said locker body for securing the tensioning members, said means having a latent condition wherein the tensioning members are movable with respect to said locker body, and an activated condition wherein the tensioning members are prevented from moving relative to said locker body.

52. The apparatus of claim 51, wherein said means includes a pin movable between a first position and a second position, said locker in the latent condition when in the first position and in the activated condition when in the second position.

53. The apparatus of claim 51, wherein said means includes a cam member rotatable between a first position and a second position, said locker in the latent condition when in the first position and in the activated condition when in the second position.

54. The apparatus of claim 51, wherein said means includes a pair of pins, at least one of the pins movable relative to the other pin so as to capture the tensioning members between said pins when in the activated condition.

55. The apparatus of claim 51, wherein said means includes at least one spring arm movable between a first position and a second position, said locker in the latent condition when in the first position and in the activated condition when in the second position.

56. The apparatus of claim 51, wherein said means includes a plurality of clamping arms movable between a first position and a second position, said locker in the latent condition when in the first position and in the activated condition when in the second position.

57. The apparatus of claim 51, wherein said means includes an expandable member expandable between a first position and a second position, said locker in the latent condition when in the first position and in the activated condition when in the second position.

58. The apparatus of claim 51, wherein said catheter assembly includes a first inner catheter and a second outer catheter, said outer catheter having an activation member rotatable relative to said inner catheter to place the locker in the activated condition.

59. The apparatus of claim, wherein said catheter assembly includes a first inner catheter and a second outer catheter, said outer catheter having an activation member axially movable relative to said inner catheter to place the locker in the activated condition.

60. A method for securing one or more tensioning members at a surgical site within the body of a patient using a catheter assembly releasably coupled to a locker, the catheter assembly having an inner catheter and an outer catheter having an activation mechanism movable relative to the inner catheter and the locker includes a movable member movable between a first position in which the tensioning members are movable relative to the locker and a second position in which the tensioning members are secured against movement relative to the locker, the method comprising:
inserting the catheter assembly into the vasculature of the patient so that the locker is proximate the surgical site;
tensioning the tension members; and
actuating the activation mechanism so as to move the movable member from the first position to the second position.

61. The method of claim 60, wherein actuating the activation mechanism includes rotating the activation mechanism relative to the inner catheter so as to move the movable member to the second position.

62. The method of claim 60, wherein actuating the activation mechanism includes axially displacing the activation mechanism relative to the inner catheter so as to move the movable member to the second position.
